(19) United States
(12) Patent Application Publication
(10) Pub. No.: US 2019/0008793 A1
(43) Pub. Date: Jan. 10, 2019

(54) INTRASITE ADMINISTRATION AND DOSING METHODS AND PHARMACEUTICALS FOR USE THEREIN

(71) Applicant: HKL Medical, LLC, New York, NY (US)

(72) Inventors: Terrence Fletcher Holekamp, East Seattle, WA (US); Lawrence Gerard Lenke, New York, NY (US); Michael Patrick Kelly, Saint Louis, MO (US)

(73) Assignee: HKL Medical, LLC, New York, NY (US)

(21) Appl. No.: 16/128,617

(22) Filed: Sep. 12, 2018

Related U.S. Application Data
(63) Continuation of application No. 15/892,227, filed on Feb. 8, 2018.
(60) Provisional application No. 62/456,642, filed on Feb. 8, 2017.
(60) Provisional application No. 62/456,639, filed on Feb. 8, 2017.

(51) Int. Cl. A61K 9/70; B01D 61/14; A61M 11/00; B01D 15/36; B01D 3/14; B01D 15/38; A61P 31/04; A61M 27/00; A61M 11/06; A61M 11/02; A61L 27/54; A61K 47/38; A61K 47/36; A61K 47/24; A61K 47/14; A61K 47/12; A61K 47/10; A61K 38/14; A61K 31/437; A61K 9/06; A61M 35/00

(52) U.S. Cl. CPC A61K 9/7015; A61M 35/00; A61M 11/007; B01D 15/361; B01D 3/14; B01D 15/3804; A61P 31/04; A61M 27/00; A61M 11/06; A61M 11/02; A61L 27/54; A61K 47/38; A61K 47/36; A61K 47/24; A61K 47/14; A61K 47/12; A61K 47/10; A61K 38/14; A61K 31/437; A61K 9/06; A61L 2300/406; B01D 61/145

(57) ABSTRACT

A new method of targeted drug administration to wounds (surgical or traumatic), intrasite (IS), offers advantages in treatment efficacy and safety over traditional routes of administration. A novel method of dosing IS medications based on wound surface area provides the parameters for safe and effective dosing, a necessary advance for any FDA approval. Large IS doses increase risk of toxicity from impurities allowed in drugs given by other routes. Methods are presented for ultrapurification, particularly of endotoxins. Methods are presented for sterile delivery to the wound, to prevent aerosolization, and to homogenize application. Pharmacodynamic parameters make certain drugs advantageous as IS agents, including slow trans-wound surface diffusion, protein binding, and limited local tissue toxicity. Vancomycin is a prototypical drug with these features and is therefore very useful as an IS medication. Other drugs, including but not limited to rifaximin, possess similar pharmacodynamics and may be useful IS pharmaceuticals, delivered alone or in combination with other drugs, carriers, or materials. All of these attributes are advantages over traditional administration methods.

### PRIORITY CLAIM

This application claims priority to U.S. Provisional Application No. 62/456,639, filed Feb. 8, 2017, and to U.S. Provisional Application No. 62/456,642, filed Feb. 8, 2017. Both of those applications are incorporated herein by reference in their entireties.

### FIELD

The present disclosure relates to the use of active pharmaceutical ingredients administered directly into wounds (surgical and/or traumatic), in order to prevent, inhibit, and/or treat disease, promote and/or improve health. More particularly, embodiments may have antimicrobial, antithrombotic, prothrombotic, antinecrotic, antiapoptotic, antineoplastic, chemotherapeutic, osteogenic, osteolytic, anti-inflammatory, analgesic, anti spasmodic, paralytic activity, prevent or promote wound healing, and/or function as growth factors or growth suppressors, among other actions.

### BACKGROUND

Traditional pharmaceutical drug administration methods like enteric ingestion (oral or rectal) or intravascular injection (IV) rely on absorption and distribution throughout the body via systemic blood circulation to achieve their effects at the site of disease. For most drugs administered in these ways, only a small fraction reaches the intended target while the majority is distributed to undiseased areas. This fact has two negative consequences: 1) reduced target site drug concentration resulting in decreased potential efficacy against disease, and 2) elevated systemic drug concentration resulting in increased potential for side-effects, systemic toxicities, and creation of drug resistant organisms.

Currently, drugs approved for the prevention and/or treatment of disease at wound sites (either surgical or traumatic), require traditional methods of administration (enteric or intravenous). These non-targeted delivery methods require drug distribution via the systemic circulation to reach the wound, reducing drug concentration at the site of disease and increasing side-effect and toxicity risks. Targeted drug delivery to wounds is needed to address these inherent problems with traditional drug administration. Wound-targeted drug delivery will increase drug effects at the wound (the site of disease), while decreasing or eliminating drug effects, side effects, and/or toxicities on non-target tissues. Specifically, with regard to antimicrobials, targeted delivery will reduce drug exposure to resident systemic microorganisms, thereby reducing the risk of creating drug-resistance. Targeted drug delivery to wounds can also allow chemicals to be safely used as drugs that would be unsafe when administered via traditional means. In these ways, targeted drug delivery methods can improve efficacy and decrease the risk of patient harm.

### SUMMARY

One method of targeting pharmaceuticals to wounds is to administer the drug by direct application into the wound itself, rather than via the systemic circulation. This new route of administration is termed “intrasite” and abbreviated “IS.” Intrasite application of pharmaceuticals constitutes both a different route of administration from traditional methods and is a form of targeted drug delivery that concentrates medication in the wound without requiring the transport-release mechanisms found in systemically-administered forms of delivery. Drugs administered intrasite obey different pharmacodynamics than drugs administered by oral, rectal, intravascular, topical, or other methods. Specifically, intrasite administration is not similar to, and obeys very different pharmacodynamics than, topical administration because a wound lacks the epidermal barrier to drug absorption into underlying tissues and the systemic circulation. Furthermore, surgical and traumatic wounds often expose multiple tissue types that can significantly alter both local and systemic pharmacodynamics. This means that intrasite drugs demand different dosing parameters for safety and efficacy than are required for the same drug administered through another route, and will require specific regulatory approval for use this new intrasite route of administration.

One aspect of this disclosure is directed to a method of administering a drug to a wound surface area of a wound in a subject, comprising administering a therapeutically effective amount of the drug to the wound surface area of the wound in the subject, wherein the drug has a low rate of absorption through tissue of the wound into a systemic circulation of the subject, is non-toxic or has low toxicity to the tissue of the wound, and remains concentrated in an amount effective to treat a condition at the wound.

In some embodiments of the method, the therapeutic agent is not absorbed into the systemic circulation. In other embodiments, the therapeutic agent is not detectable in the blood of the subject. In further embodiments, the therapeutic agent has a high affinity for protein. In certain embodiments, the therapeutic agent is bound by one or more proteins in the wound. In some embodiments, the therapeutic agent maintains a low risk of side effects.

In some embodiments, the method further comprises installing a drain in the wound.

In some embodiments, the therapeutic agent is antimicrobial. In certain embodiments, the therapeutic agent is antithrombotic or prothrombotic. In further embodiments, the therapeutic agent is antinecrotic or antiapoptotic. In still further embodiments, the therapeutic agent is antineoplastic. In yet further embodiments, the therapeutic agent is chemotherapeutic. In other embodiments, the therapeutic agent is osteogenic or osteolytic. In some embodiments, the therapeutic agent is anti-inflammatory or analgesic. In certain embodiments, the therapeutic agent is antispasmodic or paralytic. In other embodiments, the therapeutic agent is a growth factor or suppressor. In further embodiments, the therapeutic agent prevents, inhibits, or promotes healing.

In some embodiments, the wound is traumatic. In other embodiments, the wound is surgical.

In some embodiments, administering a therapeutically effective amount of the drug to the wound surface area of the wound in the subject comprises applying a thin film comprising the therapeutically effective amount of the drug to the wound surface area of the wound. In certain embodiments, the thin film comprises microcrystalline cellulose, maltodextrin, or maltotriose. In other embodiments, the thin film comprises glycerol, propylene glycol, polyethylene glycol, phthalate, or citrate.

Another aspect of this disclosure is directed to a method of administering a low bioavailability therapeutic to a wound surface area of a wound in a subject, comprising administering an effective amount of the low bioavailability therapeutic to the wound surface area of the wound, wherein the effective amount depends on at least a portion of the wound surface area of the wound to which the therapeutic is administered and wherein the therapeutic exhibits low bioavailability by not absorbing systemically to an amount sufficient to produce a systemic effect in the subject.

In some embodiments, the therapeutic inhibits growth of a target pathogen.

In some embodiments, the portion of the wound surface area is determined by measuring the length and the depth of the portion. In certain embodiments, the wound surface area is determined by scanning the wound with a device.

In some embodiments, the effective amount further depends on identifying a fraction of the wound surface area comprising adipose. In certain embodiments, the effective amount further depends on identifying a fraction of the wound surface area comprising bone. In further embodiments, the effective amount further depends on identifying a fraction of the wound surface area comprising viscera. In still further embodiments, the effective amount further depends on identifying a fraction of the wound surface area covered nervous tissue. In yet other embodiments, the effective amount further depends on identifying a fraction of the wound surface area comprising uncovered nervous tissue. In other embodiments, the effective amount further depends on identifying the rates of bleeding, transudation, or exudation. In some embodiments, the effective amount further depends on accounting for the use of a wound drain. In certain embodiments, the effective amount further depends on the use of a surgical implant.

In some embodiments, the effective amount further depends on identifying the type of wound. In certain embodiments, the type of wound is surgical. In other embodiments, the type of wound is traumatic.

In some embodiments, the effective amount further depends on whether the wound is contaminated.

In certain embodiments, the effective amount further depends on the state of closure of the wound.

In some embodiments, the effective amount is determined by identifying the fraction of the surface area comprising adipose. In certain embodiments, the effective amount is determined by identifying the fraction of the surface area comprising bone. In some embodiments, the effective amount is determined by identifying the fraction of the surface area comprising viscera. In other embodiments, the effective amount is determined by identifying the fraction of the surface area covered nervous tissue. In still other embodiments, the effective amount is determined by identifying the fraction of the surface area comprising uncovered nervous tissue. In some embodiments, the effective amount is determined by identifying the rates of bleeding, transudation, or exudation. In further embodiments, the effective amount is determined by accounting for the use of a wound drain. In still further embodiments, the effective amount is determined by accounting for the use of a surgical implant.

In some embodiments, the effective amount is administered at similar concentrations across the surface area of the wound. In certain embodiments, the effective amount is administered in a weighted manner based on at least one characteristic of the wound. In certain embodiments, the at least on characteristic is selected from the group consisting of the suprafascial nature of the wound, the subfascial nature of the wound, subcuticular edges, muscle, bone, joint, and viscera.

In further embodiments, the effective amount comprises administering a graft material comprising at least a portion of the effective amount. In certain embodiments, the graft material comprises material selected from the group consisting of an admixture with bone graft, a bone substitute, bone product, hydroxyapatite, and bone cement.

In certain embodiments, the low bioavailability therapeutic comprises vancomycin. In other embodiments, the low bioavailability therapeutic comprises rifaximin. In some embodiments, the low bioavailability therapeutic comprises a combination of vancomycin and rifaximin.

Another aspect of this disclosure is directed to a method of inhibiting an infection in a wound in a subject, comprising administering a therapeutically effective amount of an antimicrobial agent to a wound surface area of the wound in the subject, wherein the antimicrobial agent has a low rate of absorption through tissue of the wound into a systemic circulation and is non-toxic or has low toxicity to the tissue of the wound, wherein the therapeutically effective amount is sufficient to inhibit growth of a target pathogen, and wherein the concentration of the antimicrobial agent in the systemic circulation of the subject subsequent to administration is below the concentration necessary to produce an undesired systemic effect.

In some embodiments, the antimicrobial agent is not absorbed into the systemic circulation. In certain embodiments, the antimicrobial agent is not detectable in a serum sample of the subject. In further embodiments, the antimicrobial agent has a high affinity for protein. In certain embodiments, the antimicrobial agent is bound by one or more proteins in the wound.

In some embodiments, the antimicrobial agent maintains a low risk of side effects.

In some embodiments, the method further comprises installing a drain in the wound.

In some embodiments, the antimicrobial agent comprises vancomycin. In other embodiments, the antimicrobial agent comprises rifaximin. In further embodiments, the antimicrobial agent comprises vancomycin and rifaximin.

In some embodiments, the wound is traumatic. In other embodiments, the wound is surgical.

Yet another aspect of this disclosure is directed to a method of selecting a therapeutic agent for use in intrasite administration, comprising providing one or more therapeutic agents, and selecting a therapeutic agent that has one or more characteristics selected from the group consisting of low oral bioavailability, high protein-binding affinity, low or no toxicity to wound tissue, antimicrobial activity, low rate of induction of microbe resistance, low or no rate of absorption through wound tissue, and activity against biofilm.

In some embodiments, the therapeutic agent is antimicrobial. In other embodiments, the therapeutic agent is antithrombotic or prothrombotic. In certain embodiments, the therapeutic agent is antinecrotic or antiapoptotic. In further embodiments, the therapeutic agent is antineoplastic. In still further embodiments, the therapeutic agent is chemotherapeutic. In yet other embodiments, the therapeutic agent is osteogenic or osteolytic. In some embodiments, the therapeutic agent is anti-inflammatory or analgesic. In other embodiments, the therapeutic agent is antispasdmodic or paralytic. In further embodiments, the therapeutic agent is a growth factor or suppressor. In certain embodiments, the therapeutic agent inhibits or promotes healing.

A further aspect of this disclosure is directed to a system for ultrapurification of pharmaceuticals, comprising a high-throughput differential liquid filtering unit; a high-throughput fractional distillation and recrystallization unit; an detection system for detection of impurities; an automated control apparatus; an automated or controlled stopcock or manifold configured to direct fractions of filtered solvent to different destinations; and an automated or controlled stopcock or manifold configured to combine fractions of filtered solvent.

In some embodiments, the system further comprises a lyophilization unit. In certain embodiments, the lyophilization unit is temperature controlled.

In some embodiments, the detection system is in-line. In other embodiments, the detection system is out-of-line. In some embodiments, the detection system comprises technology selected from the group consisting of mass spectrometry, NMR, surface plasmon resonance, a quantitative limulus amebocyte lysate assay, and a human endothelial cell E-selectin binding assay.

Yet another aspect of the disclosure is directed to a pharmaceutical composition comprising a therapeutically effective amount of ultrapurified vancomycin, wherein the vancomycin comprises a maximum endotoxin concentration of 0.016 EU/mg. In some embodiments, the therapeutically effective amount of vancomycin is about 5 g. In further embodiments, the therapeutically effective amount of vancomycin is 10 g. In still further embodiments, the therapeutically effective amount of vancomycin is 15 g. In yet other embodiments, the therapeutically effective amount of vancomycin is 20 g. In other embodiments, the therapeutically effective amount of vancomycin is 25 g.

Another aspect of this disclosure is directed to a method of reducing aerosolization of a lyophilized pharmaceutical composition having at least one low bioavailability therapeutic agent, the method comprising wetting the lyophilized pharmaceutical composition having at least one low bioavailability therapeutic agent, wherein the wetting results in a paste but does not fully dissolve the pharmaceutical composition; dissolving the paste into a solution; emulsifying the solution with a metabolizable emulsifying agent; and creating a gel comprising the emulsified solution, a gel comprising an aqueous solvent, and a metabolizable gelling agent; wherein the gel is resistant to aerosolization.

In some embodiments, the emulsifying agent is lecithin. In certain embodiments, the gelling agent is non-proteinaceous. In further embodiments, the gelling agent is a polysaccharide gelling agent. In some embodiments, the polysaccharide gelling agent is selected from the group consisting of carbomers, poloxamers, and cellulose derivatives. In further embodiments, the gelling agent comprises pluronic, lecithin, or isopropyl palmitate.

A further aspect of this disclosure is directed to a wound treating device, comprising a dispensing pathway; a medication receptacle fluidically connected to the dispensing pathway and configured to receive a container of medication; a dosing mechanism comprising a dosing meter fluidically connected to the medication receptacle and configured to release a pre-set amount of medication into the dispensing pathway; a propellant receptacle fluidically connected to the dispensing pathway and configured to receive a container of propellant; a trigger configured to cause propellant to be released from the propellant container into the dispensing pathway; a solvent receptacle fluidically connected to the dispensing pathway; a mixing venturi nozzle, configured to mix solvent and medication to achieve particles of at least 10 μm when the trigger is actuated.

In some embodiments, the dosing mechanism comprises a plunger contained within a graduated syringe. In other embodiments, the solvent receptacle further comprises a chamber for holding at least one solvent. In certain embodiments, the solvent is ethanol. In further embodiments, the solvent is Ringer's solution. In some embodiments, the solvent is saline. In certain embodiments, the solvent comprises a gel.

### BRIEF DESCRIPTION OF THE FIGURES

Some embodiments are illustrated by way of example in the following drawings. The embodiments contemplated are not held to be limited to the embodiments depicted in these drawings.

FIG. 1 depicts a schematic of one embodiment of the process for depyrogenation/ultrapurification involving removal of endotoxins from pharmaceutical ingredients.

FIG. 2 depicts a schematic of an alternate embodiment of the process for removal of endotoxins from pharmaceutical ingredients by using a bedded polystyrene/polymyxin B filter.

FIG. 3 depicts a schematic of the process for testing filter output fractions for the presence of pharmaceutical and endotoxins.

FIG. 4 depicts a schematic for automated filter column output destination switching using two stages of machine-controlled stopcocks or manifolds.

FIG. 5 depicts a schematic of column fraction output destinations and final lyophilization of depyrogenated/ultrapurified pharmaceutical.

FIG. 6 depicts one embodiment of the method for determining intrasite pharmaceutical dosage based on manual measurement to estimate wound surface area.

FIG. 7 depicts an alternate embodiment of the method for determining intrasite pharmaceutical dosage based on automated measurement of wound surface area and tissue composition utilizing a scanning device.

FIG. 8 depicts a schematic of one embodiment of the intrasite method of administration for pharmaceutical ingredients involving manual delivery of lyophilized powder to wound surfaces.

FIG. 9 depicts a schematic of an alternate embodiment of the intrasite method of pharmaceutical administration involving a spray applicator device.

FIG. 10 depicts a partially exploded side view of one embodiments of the intrasite medication spray applicator of the present disclosure.

FIG. 11 depicts a partially exploded side view of an alternate embodiment of the intrasite medication spray applicator including several variant features.

FIG. 12 depicts a partially exploded side view of an alternate embodiment of the intrasite medication spray applicator including several variant features.

FIG. 13 is a schematic view depicting some example design variations of spray tips to meet different application needs.

FIG. 14 depicts the suprafascial wound concentrations of vancomycin at certain time intervals after intrasite administration.

FIG. 15 depicts the subfascial wound concentrations of vancomycin at certain time intervals after intrasite administration.

FIG. 16 depicts the systemic circulation serum concentration of vancomycin at certain time intervals after intrasite administration.

### DETAILED DESCRIPTION

As used herein, the term “and/or” includes any and all combinations of one or more of the associated items. As used herein, the terms “a”, “an”, and “the” mean one or more, unless contextually or specifically indicated otherwise. As used herein, unless otherwise indicated, “IV” stands for “intravenous” and “PO” stands for “per orem” and means the oral route of drug administration. “IS” stands for “intrasite”, meaning administration of drug directly into a wound. The term “IS drug” refers to drugs suitable for use in the IS administration methods disclosed herein. The terms “drug,” “pharmaceutical,” “medication,” “active pharmaceutical ingredient,” “therapeutic,” and “therapeutic agent” are used interchangeably herein, unless the context indicates otherwise.

As used herein, the term “about” means±10% of a stated value. As used herein, the term “subject” means a human or an animal. In some embodiments, a subject is a mammal. Exemplary animals include mouse, rat, rabbit, guinea pig, dog, cat, horse, cow, pig, monkey, chimpanzee, baboon, rhesus monkey, sheep, and goat. As used herein, the terms “treat”, “treating” or “treatment” refers to the reduction, amelioration, or improvement of a disease or disorder, or the reduction, amelioration, or improvement of at least one symptom of a disease or disorder, or the inhibition or prevention of the progression of a disease or disorder or a symptom of a disease or disorder. The terms “disorder”, “disease”, and “condition” are used herein interchangeably for a condition in a subject.

Unless otherwise defined, all terms (including those of a scientific and technical nature) used herein have the same meaning as would be commonly understood by one having ordinary skill in the art.

There are a number of steps and techniques disclosed herein. While each of these steps and/or techniques has individual benefit toward the final result of the process, each can be used on conjunction with one or more, or in some cases all, of the other parts of the process, and in different order from that described in the example embodiment, to achieve like results. Accordingly, for the sake of clarity and brevity, this disclosure refrains from repeating every possible combination of steps or techniques contemplated in the scope of this disclosure, to achieve like results. This disclosure should be read with the understanding that such alternate combinations are entirely within the contemplated scope of this disclosure and the claims herein.

Aspects of the disclosed methods and compositions involve therapeutic agents that are poorly absorbed systemically through wound tissues such that they do not have untoward systemic effect on a subject. For instance, a poorly absorbed agent does not absorb sufficiently across wound surfaces into systemic circulation to have a toxic effect or side effect outside of the wound in the subject to which the drug has been administered. The disclosed methods and compositions employ drugs with pharmacodynamics where systemic absorption results in systemic concentrations below those necessary to cause detectable untoward systemic effects in the subject.

Every drug is delivered to the patient via a defined “route of administration” (oral, intravenous, topical, etc.), which dictates the drug's pharmacokinetics and pharmacodynamics. Accurate dosing is affected by route-specific rates of absorption, distribution, and clearance. These parameters ultimately determine the dose-dependent rates of efficacy, toxicity, and side effects for that drug. Altering the route of administration changes that drug's pharmacokinetics and pharmacodynamics and can dramatically alter its dose-dependent safety and efficacy parameters. For this reason, national regulatory agencies like the United States Federal Drug Administration approve the use of drugs on the basis of proven safe dosing parameters via a specific route of administration. While approved drugs can be used “off-label” by varying from the initially approved indication, altering the route of administration and/or delivery outside of safe dosing parameters is forbidden in the interest of patient safety.

For the purpose of this disclosure, the term “wound” is defined as an injury to living tissue, purposeful or non-purposeful, caused by trauma or surgery, and resulting in disruption of a membrane, usually the skin, with exposure and/or injury of underlying tissues. Wounds are characterized by their location, causal mechanism, morphology in terms of length, width, and depth, tissue-types affected, degree of surrounding tissue damage, time-period of environmental exposure before treatment, and the degree of contamination with microbes or foreign material. Though it is usually the skin that is disrupted, penetrated, punctured, lacerated, or incised to create a wound, a wide variety of underlying tissues can be exposed and/or affected by the creation of a wound including, but not limited to subcuticular, dermal, subdermal, adipose, fascia, muscle, tendon, ligament, bone, cartilage, vasculature, viscera, endothelium, mucosa, neural, etc. As used herein, the terms “surface area of the wound” and “wound surface area” (WSA) refer to the measurable area of the tissue surfaces within a wound that lie deep to the epithelium of the skin or scalp.

Traditional routes of drug administration generally rely on absorption into the systemic blood circulation and subsequent distribution throughout the body for the drug to reach, and have a therapeutic effect on, the target area of disease. The consequences of this untargeted approach include time delays to the onset of drug effect, reduced target site drug concentration (and therefore reduced effect per dose), and increased systemic drug concentration resulting in potential off-target toxicities and side effects. This can be particularly problematic when administering systemic drug doses to treat target wound tissues (either surgical or traumatic in origin), due to natively poor and/or disrupted circulation in these areas. Such problems with non-targeted drug administration have made initial treatment failures common when managing wound-related diseases like infections (the most common serious adverse event associated with wounds), often requiring prolonged systemic (enteric or IV), antibiotic treatment, multiple surgical debridements, prolonged hospital stays, extreme expense, and significant morbidity or even mortality as the final outcome. This is especially true in the context of infection by drug resistant organisms. Furthermore, reduced target site drug concentration contributes to increases in organisms' development of resistance. New methods of wound-targeted drug administration that can be safer, more effective, and may make unused or underutilized medications highly effective would carry great benefits to treat these ongoing serious and urgent patient threats.

### 1) Intrasite Administration

This disclosure provides a new and highly practicable method of targeted drug administration to wounds through the placement of therapeutic agents directly into the wound, bypassing distribution via the systemic circulation. This new route of administration is referred to herein as “intrasite” and is abbreviated “IS.” Pharmaceuticals for IS administration (also referred to herein as “IS pharmaceuticals,” “IS drugs,” etc.) possess a different set of chemical properties from those needed for most IV, oral or topical medications. Furthermore, safe and effective IS administration of pharmaceuticals comprises a new understanding of pharmacodynamics and new methods of dosing based on wound surface area with potential adjustment for wound tissue-type and composition.

Drugs administered enterally and parenterally are usually absorbed into the blood stream and then circulated to the site of disease as well as the rest of the body. This results in distribution of the drug throughout the body, resulting in potential toxicity to organs, diminished drug concentration at the site of disease, and increased drug resistance by resident microorganisms. When medications are delivered through the bloodstream, dose-dependent rates of efficacy and toxicity are directly associated: increasing drug dose may increase efficacy but simultaneously increases the risk of systemic toxicity. Drugs used for IV or oral administration (with certain exceptions) are usually sought out for chemistry allowing easy absorption/dissolution into the blood stream (also known as “high bioavailability”). The IS methods disclosed herein are counterintuitive to the prevailing notions of drug delivery and high bioavailability. In fact, in some embodiments, the IS administration of this disclosure employs medications with chemistry resulting in low bioavailability. The IS administration methods disclosed herein use drugs that do not easily absorb into systemic circulation through contact with wound tissues, instead remaining at higher concentration for a longer time within the wound. Additionally, if a drug does not enter the circulation from the wound easily, the systemic concentration remains low, minimizing the risk of systemic toxicities and side effects. Therefore, with favorable chemical properties, a smaller total dose of medication can be administered IS, resulting in higher wound drug concentration (and therefore higher local efficacy), and lower systemic concentration (thereby reducing risk of systemic toxicity), than is possible with conventional IV or enteric administration. Also, in contrast to topical administration, which may allow only limited or unpredictable drug penetration below the epidermis, IS administration places the drug in direct contact with underlying wound tissues. This fact allows for greater predictability and accuracy in dosing to account for the pharmacodynamics of the specific tissue(s) being treated.

Topical and the IS routes of drug administration may be confused or conflated. However, the topical and IS routes of drug administration obey different pharmacodynamics, safety, and dosing parameters. The United States Federal Drug Administration defines “topical” as “administration to a particular spot on the outer surface of the body.” Generally, such application is to the epidermal layer of the skin or scalp. This is important because the epidermis presents a significant barrier to systemic absorption of a drug. This has allowed some drugs to be approved for topical use that are too toxic for enteral or parenteral administration (e.g., neomycin). With topical administration greater or lesser bioavailability of a drug is often not a primary pharmacodynamic concern because absorption is restricted by the epidermis. Even so, when applied to sizable defects in the epidermis like a wound, some topical medications (e.g., neomycin), are known to present systemic toxicity risks due to increased diffusion into the circulation. The pharmacodynamics of topical drug application onto the epidermis are fundamentally different from IS administration directly into traumatic or surgical wounds wherein absorption into the systemic circulation is a primary pharmacodynamic concern that must be addressed for patient safety.

Some embodiments of the disclosed methods and compositions include modifying a therapeutic agent that is bioavailable through other routes of administration to render it poorly bioavailable such that the therapeutic agent administered IS does not absorb, or has a low rate of absorption, to the systemic circulation, organs, or internal tissues (i.e., rendering the therapeutic agent a low bioavailability therapeutic agent). For instance, a compound can be modified to make it less soluble, increase the size of the compound, increase the “stickiness” of the compound, and alter the hydrophobicity of the compound. In some embodiments, therapeutic agents are modified through chemical conjugation. While there will be many bioactive chemicals and/or known drugs that are not good candidates for IS administration due to absorption through the wound into the systemic circulation, many of these may be transformed into IS medications with very favorable pharmacodynamics, through conjugation to larger molecules. In the case of small molecule drugs, conjugation to long chain carbohydrates like dextrans, or the like, is one way of accomplishing a reduction in the rate of tissue diffusion without impairing the mechanism of action. For antimicrobial peptides (discussed in more detail below), conjugation to dalargin-polyethylene glycol, or the like, may present both a means of enhancing biological effects as well as preventing diffusion. It is contemplated within the scope of this disclosure that conjugation techniques of this kind can transform some chemicals into useful IS pharmaceuticals by improving their pharmacodynamics profile. It should be noted that any modifications are acceptable so long as the modification results in sufficient decreases to the absorption profile of a therapeutic agent so that the therapeutic agent is not absorbed into internal tissues, organs, or the systemic circulation of a subject in an amount to cause an undesired or untoward effect or any other side effect.

In other embodiments, an IS medications can be loaded into hydrogel polymers prior to, or at the time of, administration. A drug-loaded hydrogel with favorable properties can release drug onto wound tissues and into seroma fluid more slowly than applying the drug directly, flattening the drug concentration curve over time. Such drug-loaded hydrogels can have the dual benefits of maintaining effectively high drug levels longer and lowering the concentration gradient for diffusion into the circulation, particularly in the time immediately after the dose is given. Drug-loaded hydrogels of this kind may be useful as coatings for implanted materials, a matrix for aggregation of grafted tissues or materials (e.g., bone or bone substitutes), or administered directly to tissue surfaces in the form of a spray. Exemplary devices for spray application of IS medications, potentially with hydrogel carriers, are discussed in detail below. Examples of potentially suitable hydrogel formulations include, but are not limited to, photo-crosslinkable oligo(poly(ethylene glycol)fumarate)/sodium methacrylate copolymer, chemo-crosslinkable polyaldehyde dextrans, photo-initiated chemo-crosslinkable poly(N-isopropylacrylamide)-poly(vinylpyrrolidinone), and the like. It is contemplated within the scope of this disclosure that conjugation techniques of this kind can transform some chemicals into useful IS pharmaceuticals by improving their pharmacodynamics.

Intrasite drug administration is a superior way to target drug effects to the wound by maximizing drug concentration at the site of disease. Furthermore, drug concentration in the systemic circulation is lower than with traditional routes of administration, due to reversed drug distribution dynamics. This reversal improves drug efficacy at the site of disease and lowers the potential for systemic toxicity and side effects (thereby improving safety).

Additionally, IS administration carries numerous other important benefits over traditional routes of administration. For one, IS administration can result in significant treatment cost reduction due to potential one-time dosing. Another benefit is the reduced potential for development of drug resistance to IS antimicrobials due to higher wound site drug concentrations (resulting in higher kill efficiency), and reduced exposure of systemic microorganisms to the drug. A third benefit of IS administration is the potentially improved therapeutic index for any given drug.

The IS route of administration of this disclosure uses low bioavailability drugs and drugs modified to have low bioavailability. In the context of IS administration and IS drugs, low bioavailability means a drug that absorbs poorly, slowly, or not at all through tissue of a wound into systemic circulation. Such drugs are advantageous due to a natural tendency to remain concentrated within the wound and absorb poorly/slowly into the systemic circulation. This is counter-intuitive to current practices and typical routes of administration. In traditional routes of administration, low bioavailability ordinarily makes a chemical unsuitable as a medication when delivered via traditional routes of administration because these drugs will not reach sufficient concentrations to allow for a therapeutic effect. In contrast, when used in the IS administration of this disclosure, the special advantages of low bioavailability in IS administered drugs (i.e., the drugs being characterized by their slow, poor, or non-existent absorption through wound tissue into systemic circulation) allow unused or underutilized chemicals to become safe and effective pharmaceuticals when delivered via the IS route.

The IS route of administration, methods of dosing, methods of delivery, and methods of purification and drug modification disclosed herein can be used for drugs of many purposes, including antimicrobial, antithrombotic, prothrombotic, antinecrotic, antiapoptotic, antineoplastic, chemotherapeutic, osteogenic, osteolytic, anti-inflammatory, analgesic, antispasmodic, paralytic, prevent/promote healing, growth factor/suppressor, among other things.

The IS administration methods of this disclosure are based on a newly discovered theory and experiment-backed understanding of IS pharmacodynamics. This new theory has also led to novel methods of pharmaceutical dosing, as well as methods for identifying IS drugs with the highest utility and lowest safety risks based on pharmacodynamics and chemical characteristics of the drugs. Drugs that work with the IS administration methods disclosed herein have one or more of the following features 1) the ability to remain concentrated within the wound for extended periods of time after a single dose application, 2) a low rate of absorption through wound tissues into the systemic circulation, 3) non-toxic or low toxicity to local tissues even at high concentration, or 4) no or low rates of local or systemic off-target or side-effects. In some embodiments, the IS administration methods disclosed herein employ a drug or drugs that have all of the foregoing features. In other embodiments, the IS administration methods disclosed herein with employ a drug or drugs that have two of foregoing features. In other embodiments, the IS administration methods disclosed herein with employ a drug or drugs that have three of foregoing features. In other embodiments, the IS administration methods disclosed herein with employ a drug or drugs that have one of foregoing features.

In some embodiments, when administered by the IS methods of this disclosure, a drug with low oral bioavailability will usually absorb poorly through wound tissues into the systemic circulation and remain concentrated within the wound for extended periods after a single dose application. In other embodiments, the IS administration methods of this disclosure employ drugs which tend to bind to proteins. While this is often counterproductive for systemically administered drugs, when given IS, protein binding can improve drug pharmacodynamics. This is due to the spatial anchoring effects of binding exposed structural proteins within a wound (thereby slowing diffusion into surrounding tissues and circulation system), but also because this protein-bound fraction can act as a reservoir to extend the period of effective drug concentration within the wound. Because of these effects on IS pharmacodynamics, drugs with poor bioavailability, higher degrees of protein binding, and poor tissue penetration when administered systemically, make excellent candidates for IS pharmaceuticals.

While surgical wounds are discussed by way of example throughout this disclosure, the IS pharmaceuticals, methods, and devices disclosed herein are applicable and beneficial to traumatic wounds as well. Traumatic wounds have higher degrees of irregularity, complexity of tissue damage, and in some cases contamination or penetration by foreign material. In particular, wounds caused by high-energy projectiles or blasts, as are common in war, involve not only irregularity, complexity, and contamination, but are frequently compounded by high-pressure cavitation injury to surrounding tissues, which disrupts small vessel circulation. Due to this, projectile and blast wounds are at high risk for infection, ischemia/necrosis, and poor or delayed healing. In addition, aside from infection with a variety of bacteria, blast-related war wounds are especially high-risk for infection with aggressively invasive fungi that can be very difficult to treat with systemic antifungals. Furthermore, the small vessel disruption that accompanies high-energy mechanisms of injury make traditional circulatory methods of drug delivery prone to treatment failure. This disclosure provides IS pharmaceuticals, devices and methods of administration that address this problem through direct application to affected tissues, bypassing the need for circulatory distribution of drugs. Such IS pharmaceuticals, devices, and methods can be especially useful for treating traumatic high-energy war wounds.

2) Managing and Avoiding Consequences of IS Drug Persistence within Wounds

IS administered drugs that are not broken down within wounds or absorbed into the circulation may form persistent osmotic gradients, drawing extracellular fluid into the wound cavity. In some instances, this could cause a pressurized seroma to develop within a closed wound, potentially leading to delayed healing or wound dehiscence. In some embodiments, wound drains are placed into the wound to remove excess fluid entering the wound. Alternatively, sterile needle tap procedures, performed once or several times after wound closure, can be performed in certain embodiments to remove seroma fluid and drug from the wound at a specified time interval after wound closure. In some embodiments, a portion or entirety of the wound can be left unclosed to allow drug and seroma fluid to escape. In such instances, removal of drug and seroma fluid can be actively assisted by a negative pressure dressing. In other embodiments, a slow acting compound or enzyme that actively breaks down the IS administered drug over time is concurrently administration with the IS drug. In these instances, the concurrently-administered compound or enzyme, as well as the metabolites it creates, then breaks down or absorbs away to avoid creating an osmotically pressurized seroma.

### 3) Vancomycin as an Exemplary IS Pharmaceutical

In some embodiments, the IS administration methods described herein comprise vancomycin. Vancomycin's extremely low oral bioavailability, high degree of protein binding, and lack of local tissue toxicities are one example of a therapeutic agent profile that can be used in the methods disclosed herein. Other similar profiles can be determined by using molecular modeling tools (e.g., Kumar et al. (2011) J Nat Sci Biol Med. 2(2): 168-173)(incorporated by reference in its entirety). In addition, tissue models can be used to determine whether a therapeutic agent has the proper low bioavailability profile. Testing of bioavailability can also be performed in vivo in model organisms such as rats, mice, pigs, and dogs. Clinical tests on humans can also determine bioavailability of a particular therapeutic agent. Such in vivo testing typically involves oral or topical administration of an agent and isolating blood samples every 30 minutes to one hour. The samples are then tested to determine the concentration of the agent in blood over time to determine the Tₘₐₓ and Cₘₐₓ of the agent. However, there are multiple potential safety issues that previous research failed to recognize or address which make currently sold formulations of vancomycin unsuitable for IS administration. The first of these issues is the presence of endotoxins in all current formulations of vancomycin, a consequence of the manufacturing process. Endotoxins are very potent pyrogens, responsible for inducing sepsis syndrome, and even minute amounts absorbed through a wound into the circulation could be hazardous to patients. A second major safety concern is the “dustiness” of lyophilized medications, causing them to aerosolize with minimal perturbation. Aerosolized vancomycin, for example, is easily inhaled and presents a safety problem in the form of a known risk of inducing pulmonary fibrosis. This problem and potential solutions for drugs with these safety issues are discussed below.

### 4) Endotoxin Issue

Endotoxins are a group of lipoglycan cell wall components found on Gram-negative bacteria which are very potently toxic to humans, even in miniscule concentrations. Endotoxins cause rapid activation of immune and inflammatory cascades resulting in fever, blood vessel dilation and leakage, clotting abnormalities, shock, and sepsis syndrome. Some endotoxins can cause direct organ damage including of the kidneys, intestines, liver, and hearing apparatus. One major drawback of the manufacturing methods used to produce all current forms of vancomycin is that a small concentration of endotoxins and other impurities remain with the antimicrobial compound. These endotoxins and impurities are reconstituted and administered to patients along with the active drug. These concentrations are small enough to be considered safe by the U.S. Food and Drug Administration, provided that dosing limit guidelines for intravenous and oral administration are obeyed so as to limit the amount of endotoxins absorbed into a patient's system. Therefore, these small concentrations of endotoxins limit the doses that patients can receive safely.

Currently, the FDA allows up to 0.16 Endotoxin Units (EU)/mg in lyophilized preparations of vancomycin intended for IV route of administration. This is based on an experimentally determined IV limit of 5 EU/kg/hr as the minimal endotoxin dose rate that causes symptomatic endotoxemia in humans. For IV administration, vancomycin is meant to be infused slowly, over 1 hr, and the “normal” human for purposes of calculation is assumed to be 80 kg. Additionally, the largest safe single dose of vancomycin recommended by safety regulations is 2500 mg. Therefore: (5 EU×80 kg)/2500 mg=0.16 EU/mg vancomycin

Even the minute concentrations of endotoxins allowed in IV preparations are not considered safe by the FDA when higher doses of the drug are used. To administer vancomycin by the IS administration methods disclosed herein, higher single doses than 2500 mg can be administered to cover large wound surface areas at concentrations that are reliably bactericidal for drug resistant organisms. Therefore, safe limits of endotoxins must be lower than 0.16 EU/mg in preparations of vancomycin intended for IS administration. It is estimated that a 10-fold lower limit for endotoxin (0.016 EU/mg) will prevent conceivable IS doses of vancomycin from exceeding the 5 EU/kg/hr toxicity limit. In certain embodiments, up to 25 g single doses are administered without high risk for endotoxemia.

In view of the toxicity of endotoxins, ultrapurification of vancomycin and other IS antimicrobials is a goal of composition development, in order to allow them to be safely used at higher doses without risking toxicity related to impurities, especially endotoxins, to patients. A further need exists that these new purification processes allow for high-throughput in order to produce sufficient quantity to meet medical need. Finally, there is a need that this purification technique be inexpensive so as to maintain the cost-effectiveness of treatment.

### 5) Ultrapurification of Drugs

This disclosure provides a number of ultrapurification methods with various steps and techniques. While each of these steps and/or techniques has individual benefit toward the final result of the process, each can be used on conjunction with one or more, or in some cases all, of the other parts of the process, and in different order from that described in the example embodiments, to achieve the desired results. Accordingly, for the sake of clarity and brevity, this disclosure will refrain from repeating every possible combination of steps or techniques contemplated in the scope of this disclosure. This disclosure should be read with the understanding that such alternate combinations are entirely within the contemplated scope of this disclosure and the claims herein.

New ultrapurification techniques for pharmaceuticals, including vancomycin, are disclosed herein. It will be evident to one of ordinary skill in the field that modifications to one or more of these details can achieve like results. Further, the methods disclosed herein can be used with various pharmaceuticals. Therefore, the present disclosure is not intended to be limited to the specific details and/or embodiments depicted by the figures or description herein.

In one embodiment of a process for elimination of endotoxins from vancomycin preparations, Amycolatopsis orientalis (the organisms that produce vancomycin) is cultured through fermentation under conditions that disallow gram negative organisms to co-exist in culture, avoiding production of endotoxins in the culture medium. In one embodiment, Amycolatopsis orientalis is fermented in the presence of polymyxin, which is selectively and potently bactericidal to gram-negative organisms. In this embodiment, polymyxin concentration would be high enough to fully suppress gram-negative bacterial growth. In addition to vancomycin, this process used with other pharmaceuticals whose microbiological production can be contaminated by endotoxins from Gram-negative bacteria. In addition, polymyxin b is known to bind strongly and selectively to endotoxin and any remaining trace quantities of endotoxin in the fermentation broth can be removed by using known methods of separating vancomycin or other antimicrobial from polymyxin/endotoxin complex. Such methods for separation include, but are not limited to, high pressure liquid chromatography, fractional distillation, recrystallization, antibody pulldown, or reverse osmosis.

Another embodiment of a process for removal of endotoxins from preparations of vancomycin makes use of polymyxin as a selective and potent binding agent for endotoxin. In this embodiment, polymyxin B is covalently bonded to polystyrene threads which are packed into a filter housing. Reconstituted lyophilized vancomycin or wet-body vancomycin dissolved in aqueous solution is passed through the filter, thereby being selectively reduced of endotoxins in the process.

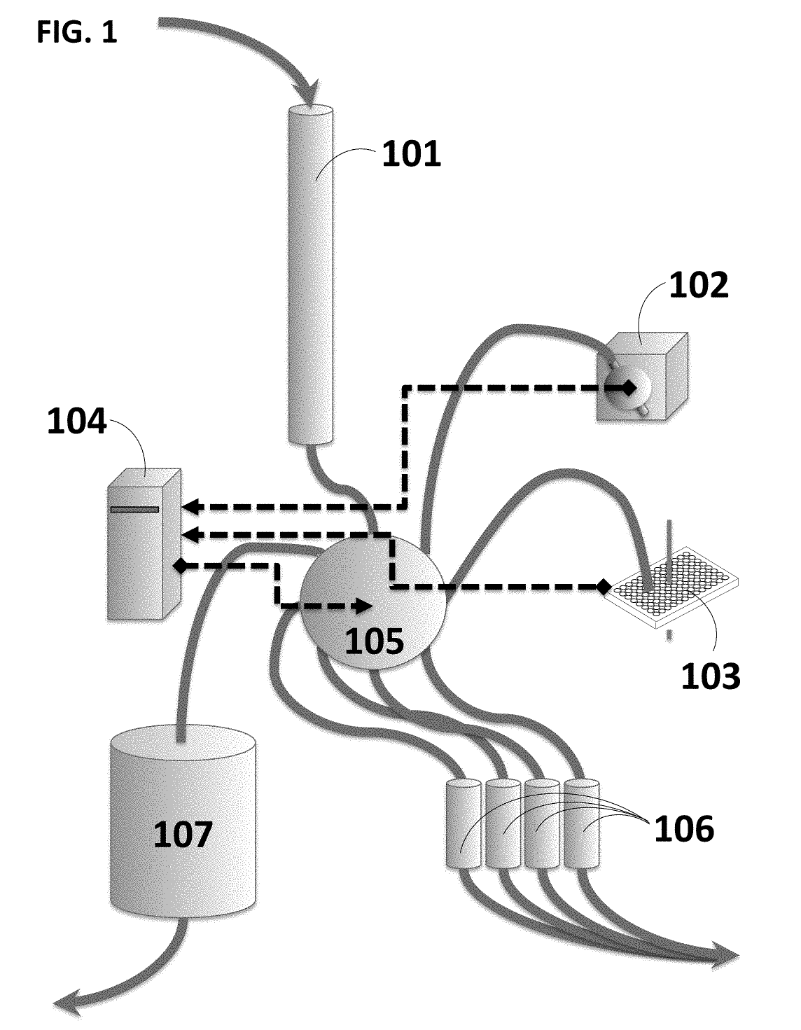

FIG. 1 depicts a schematic of one embodiment of a system for ultrapurification of intrasite pharmaceuticals, including vancomycin. In some embodiments, control of the steps in the process is automated. In other embodiments, the control of the steps is not automated. To accomplish ultrapurification, standard lyophilized vancomycin (or other pharmaceutical), is dissolved in solution and passed through filter column 101. The solvent and filter media are chosen specifically to create differential affinities for endotoxins versus pharmaceutical (e.g., vancomycin) so as to separate them in liquid phase as they pass through the filter column. In some embodiments, a pump produces pressure to drive the solvent through the filter column at a greater rate. In the embodiment of FIG. 1, the solvent is drawn through the filter by gravity. In some embodiments the filter is an ion exchange chromatography column. In some embodiments the filter is an ultrafilter designed to separate based on the molecular weight differential between endotoxins (usually >10 kDa) and active pharmaceuticals (e.g., vancomycin <1.5 kDa). As the solvent leaves the filter column, it passes into a first-stage machine-controlled stopcock or manifold valve 105. This switching valve directs a small amount of the fluid, at intervals, to testing equipment 102. In some embodiments, the testing equipment comprises a mass spectrometer to determine the presence of the vancomycin or other pharmaceutical ingredient. Data from mass spectrometer readings are then fed back to the control computer 104 for analysis. In some embodiments, detection methods other than mass spectrometry are used. In some embodiments, amplifying colorimetric assay reactions coupled to a spectrophotometer reading can be used. A variety of detection methods can also be utilized including nuclear magnetic resonance, Raman spectroscopy, Fourier-transform spectroscopy, ultraviolet-visible spectroscopy, tandem mass spectrometry, surface plasmon resonance, etc. The choice of detection method is dependent on the particular chemical/pharmaceutical assayed, sensitivity requirements, and process efficiency requirements.

Solvent fluid in which no pharmaceutical ingredient (e.g., vancomycin) can be detected is directed to a waste tank 107 by a signal sent from the control computer 104 to the first-stage control valve 105. Solvent fluid in which a pharmaceutical ingredient can be detected is retained and divided into fractions by direction toward serial fraction holding tanks 106. These pharmaceutical-positive fractions remain in holding until testing for endotoxin is completed. At intervals (in some embodiments, the interval is the moment after switching to a new fraction holding tank), a small amount of solvent fluid is directed toward the endotoxin-testing device 103. In some embodiments, this switching process is automated by computer control of the first-stage valve 105. In some embodiments, endotoxin testing is performed by an automated multi-well plate reading device 103, utilizing colorimetric or fluorescent endotoxin assays. In some embodiments, kinetic turbidimetric assays, kinetic colorimetric assays, human endothelial cell bioassay, tandem mass spectrometry, or other means are utilized for their high sensitivity.

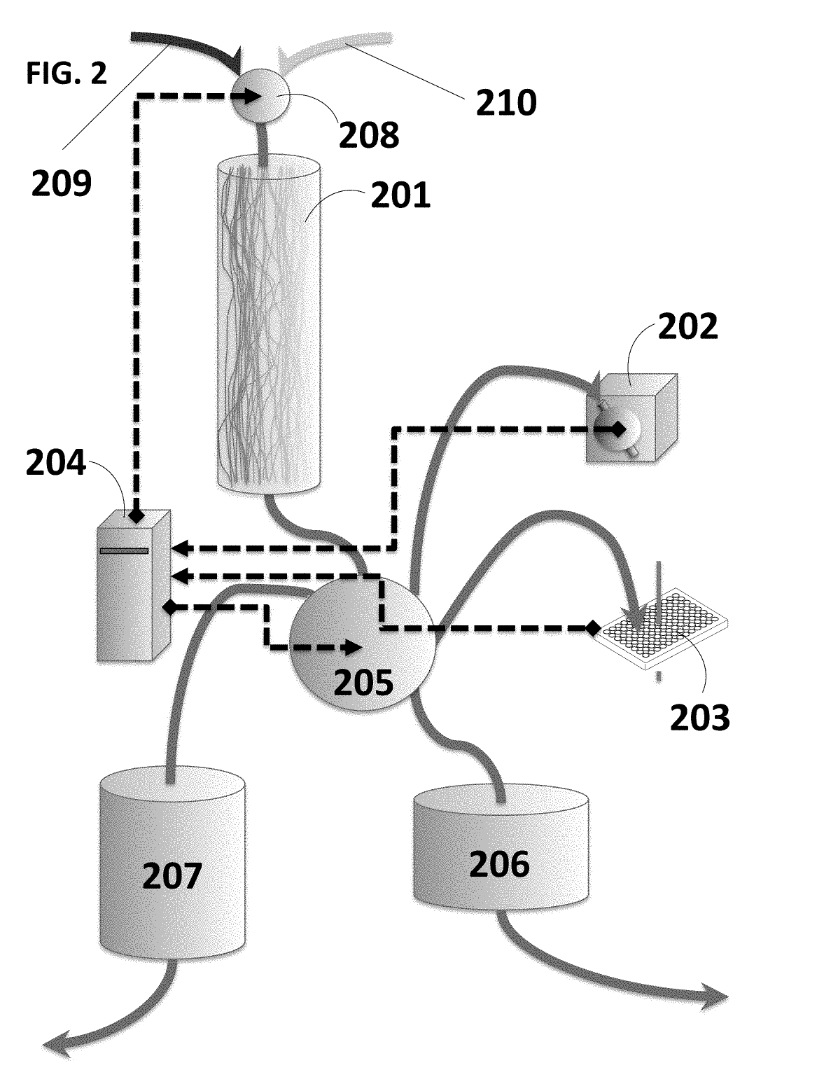

FIG. 2. depicts an alternate embodiment of a process for removal of endotoxins from preparations of pharmaceuticals which makes use of affinity sorbents like polymyxin B as a selective and potent binding agent for endotoxin. As in the depiction of FIG. 1, processes are shown automated by can be performed without, automation. In this embodiment, polymyxin B (or other affinity sorbent like L-histidine, poly-L-lysine, or poly(γ-methyl L-glutamate), is covalently bonded to polystyrene threads or other media like sepharose 4B (purple threads in the FIG. 2) which are packed into a filter housing 201. Reconstituted lyophilized vancomycin or wet-body vancomycin dissolved in aqueous solution 209 is introduced via a controllable stopcock 208, and passed through the filter 201, thereby being reduced of endotoxins by selective binding of endotoxin to bedded polymyxin B within the filter. The output fluid from the filter passes through a second controllable stopcock or manifold valve 205, which is controlled by a computer with an interface 204. Small volumes of filter output fluid are periodically sent to detection devices for pharmaceutical 202, and endotoxin 203 detection, and the results returned to the control computer 204. Filter output fluid containing no pharmaceutical or containing endotoxin is directed toward the waste tank 207, whereas fluid containing detectable pharmaceutical but no detectable endotoxin is directed toward the holding tank 206 by controlled switching of the output valve 205.

In some embodiments of the process depicted in FIG. 2, once the polystyrene/polymyxin B filter 201 is fully loaded/saturated with endotoxin, it is discarded and a fresh filter installed. In some embodiments of this process, when the filter is saturated with endotoxin, a wash solution 210 is introduced into the filter 201 by switching of the input valve 208 by the control computer 204. This wash solution 210 is intended to remove endotoxins from the saturated filter 201 so that it can be reused, thereby reducing cost and improving process efficiency. This wash solution 210 may be an alcohol (ethanol, isopropyl alcohol, phenol, etc.), a detergent/surfactant (Triton-X, Zwittergent, octyl-β-D-glucopyranoside, etc.), a high-pH solution like sodium hydroxide, an alkanediol with cationic support, or potentially other solvents able to separate endotoxin from polymyxin B without causing filter degradation. The control computer 204 directs this wash solution to the waste tank by switching output valve 205. Following this step, the endotoxin removal wash residue is removed from the filter by running pharmaceutical carrier solvent through the filter into the waste tank until wash residue is no longer detectable. By this method, the sorbent filter is recharged and returned to its original state.

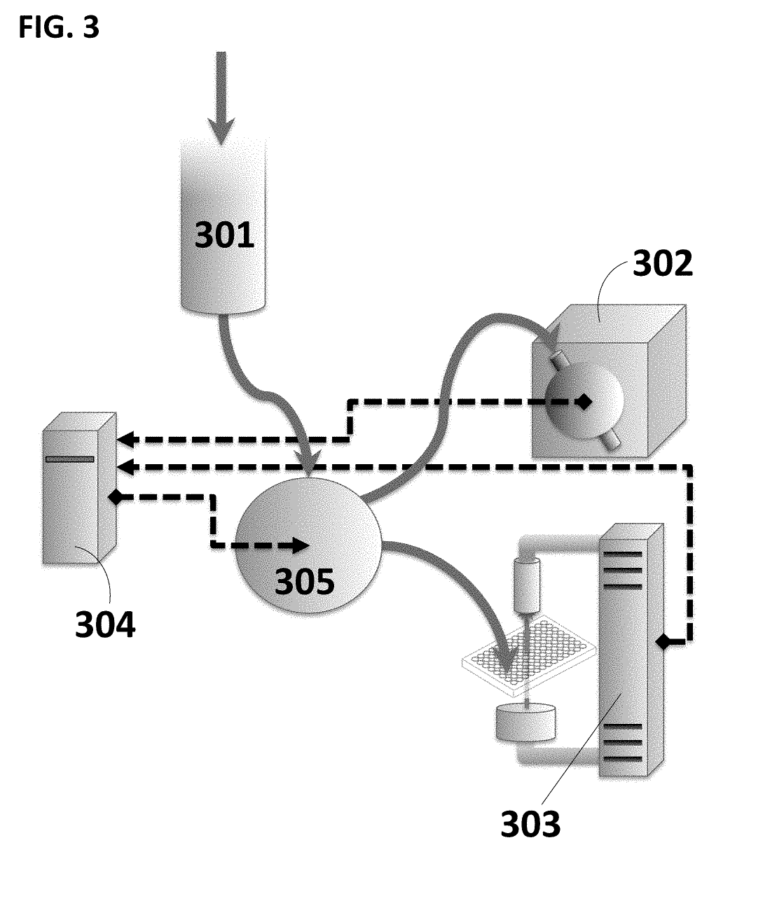

FIG. 3. depicts an exploded view of the parts of an embodiment of a system for directing solvent fluid from the filtering column 301 toward the mass spectrometer (or similar detector) 302 for pharmaceutical ingredient (e.g., vancomycin) detection, or toward the endotoxin testing device 303. Samples of filtered solvent fluid are sent at intervals to each of these testing devices by switching the filter-output control valve 305. In some embodiments, these valve adjustments are made by a control computer 304, running purpose-built software. In some embodiments, the endotoxin testing device 303, is automated to allow each new sample to be injected into a new well on the plate with a time-address recorded so that each sample can be traced to a specific fraction. Data from both testing devices (302 and 303) are fed back to the control computer 304, to provide information for process control. As in FIG. 1, control of these processes can be automated or non-automated.

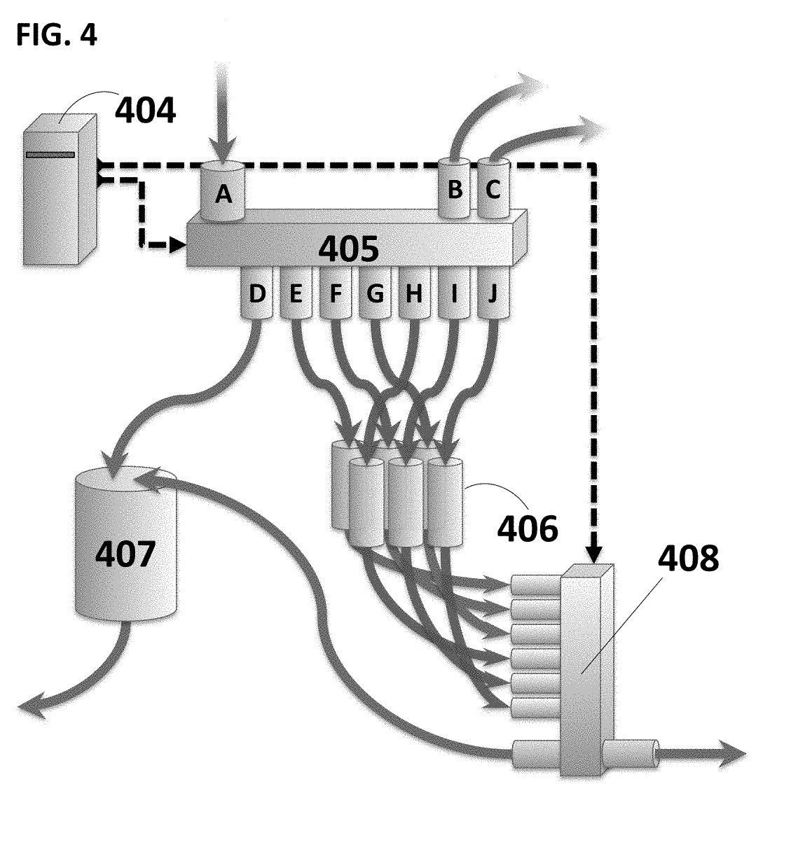

FIG. 4. depicts a more detailed view of an exemplary embodiment of a process for controlling filter column output fraction destination by adjustable valves in two stages 405 and 408. In some embodiments, as shown here, these valves are electronically-actuatable solenoid pinch valves mounted to a manifold. FIG. 4 introduces a second-stage control valve 408, in addition to the first-stage control valve 405. The second-stage control valve 408 allows for controlled recombination/pooling of the fractions, which contain ultrapurified pharmaceutical (e.g., vancomycin), from the fraction holding tanks 406. Additionally, this second manifold 408 allows controllable venting of those fractions found to contain detectable endotoxin to the waste tank 407. Designs with manifold-mounted solenoid pinch valves have the advantage that each valve is independently switchable, allowing input and output to the valve system to be set in any configuration including all on or all off. This enables filter output samples to be sent to the two testing devices (not depicted here), simultaneously by opening Valves A-C while keeping other valves closed. Alternatively, this design also allows test sample extraction “in-flight”, while simultaneously capturing fractions into the fraction holding tanks 406 (by opening Valves A, C, and E, for example). Additionally, this design prevents solvent fluid from directly contacting/contaminating the valves themselves since fluid stays inside the pinch tubing at all times. In case of a contamination event, the tubing can be changed without replacing expensive valves, an advantage for keeping production costs low. In this design configuration, system automation with a control computer 404 for the manifold pinch valves 405 and 408 are used due to the technical difficulty presented by manually switching multiple valves simultaneously.

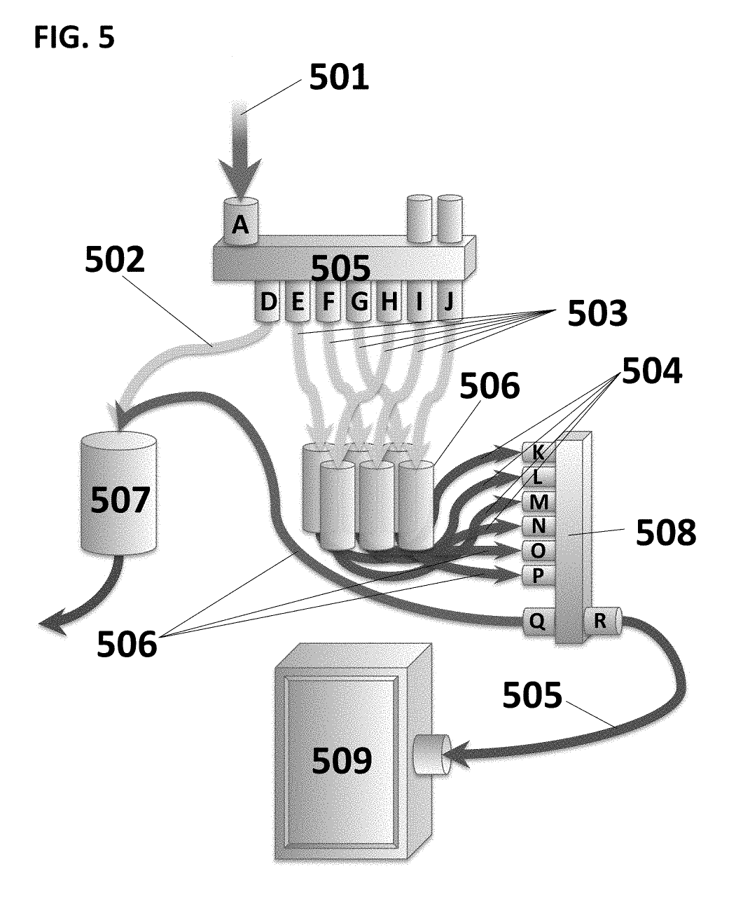

FIG. 5. depicts an exemplary embodiment of filter column fluid fraction destinations and final processing of ultrapurified pharmaceutical (e.g., vancomycin) by lyophilization. In this embodiment, filter column output fluid (arrow 501), leaves the filter and enters the first-stage control valve manifold 505, at Valve A. Filter column output with no detectable pharmaceutical (e.g. vancomycin), (arrow 502), is vented to waste tank 507 by opening Valve D. Filter column output fluid with detectable pharmaceutical is shunted (arrows 503) into serial fraction holding tanks 506, by individually and sequentially opening Valves E-J. Once endotoxin testing has been completed, filter column output fluid containing ultrapurified pharmaceutical and no detectable endotoxin (arrows 504), is recombined/pooled and passed (arrow 505) into the temperature-controlled lyophilization chamber 509, by opening Valves K, L, M, N, and R on the second-stage control valve manifold 508. Fractions shown to contain endotoxin are vented (arrows 506), to the waste tank 507 by opening second-stage Valves O, P, and Q. In some embodiments, the tubing used to conduct fluids during all parts of this system is made from a medical grade non-permeable, non-stick, low-residue substance like polytetrafluoroethylene (PTFE), or the like.

While certain materials in an example embodiment have been described herein, the ultrapurification process contemplated is not limited to these materials and other embodiments may utilize other materials to achieve like results. The selection of variant processes or materials to be used can be dictated by costs and efficiencies of components or sub-processes and can change over time to accommodate changing economic conditions. This potential need for change is contemplated to be within the scope of this disclosure.

Furthermore, it will be readily apparent to those of ordinary skill in the art that other embodiments may perform similar functions and/or achieve similar results. All such equivalent embodiments are within the spirit and scope of the present disclosure. Furthermore, the process described herein is designed to be applicable for the ultrapurification of pharmaceuticals intended for IS administration including vancomycin, rifaximin, tobramycin, etc. in order to remove toxic impurities (including endotoxins). The ultrapurification methods of this disclosure result in pharmaceuticals suitable for use in the IS route of administration methods of this disclosure.

### 6) IS Pharmaceutical Dosing Methods

As previously stated, because the IS administration methods disclosed herein involve application of pharmaceuticals directly to the tissues within a wound (either surgical or traumatic), the pharmacodynamic behavior of the medication(s) applied will be determined, in part, by interactions with and the potential for absorption through those tissues. The concentration of medication in contact with a tissue surface, as well as its absorption rate per unit surface area of the specific tissue type(s) the drug contacts, are the primary determinants of the total concentration vs. time curve of drug absorption into the circulation. These factors are also directly associated with drug efficacy as they determine drug concentration area under the curve within the wound over time and in turn the probability of both desirable and undesirable local effects. Therefore, the dosing of IS medications is calculated primarily on the basis of wound surface area. Importantly, in some instances, modifications to dosing may be necessary based on the specific tissue composition of the wound (fractional surface area comprised of muscle vs. adipose vs. bone, etc.), because different tissue types may possess different diffusion constants for the drug or its impurities.

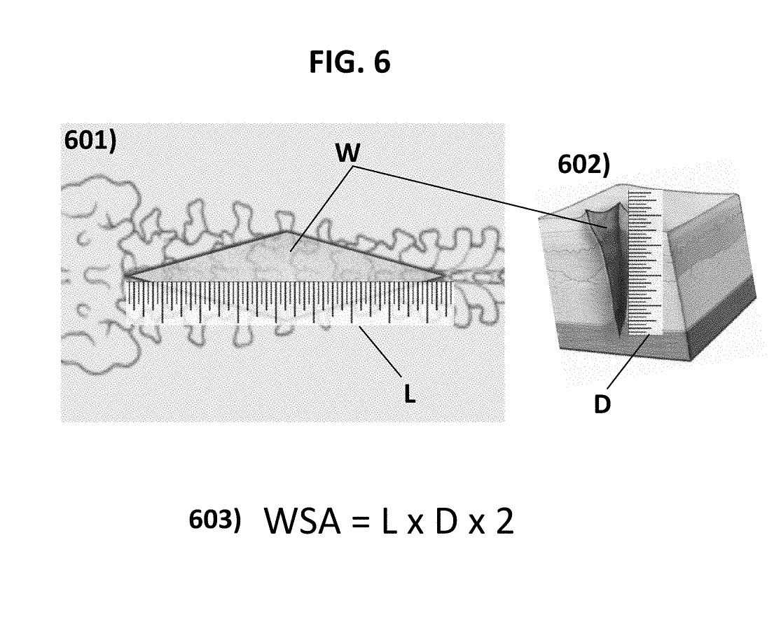

FIG. 6 depicts an exemplary embodiment of a method to calculate IS pharmaceutical dosing based on wound surface area (WSA). This exemplary method applies to surgical and traumatic wounds and involves an estimation of the WSA. In some embodiments, manual measurements of length L (seen in top down view of exemplary wound 601), and depth D (seen in side view of exemplary wound 602), of the wound W are taken with a sterile measuring device. The basic formula for estimating WSA is shown 603. From this estimated WSA, a total dose of IS medication can be calculated on the basis of dose per cm² of the WSA. In some embodiments, measurement of wound surface area can require multiple length and/or depth measurements, with averaging applied, to account for irregular wound shapes. This manual method of estimating wound surface area is less accurate when applied to wounds with high degrees of irregularity, as in trauma.

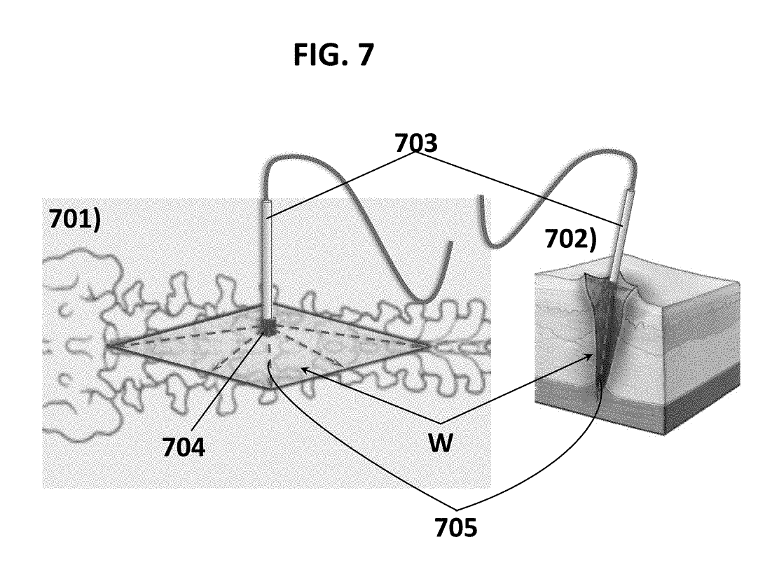

FIG. 7 depicts an alternate embodiment of a method to measure WSA using a scanning device 703. In some embodiments, the scanning device can be based on a laser 704 (or non-coherent emitter), which emits low intensity, potentially nonvisible photons (arrows 705), and uses time-of-flight and/or interferometry to measure distance from the probe to all wound surfaces (depicted in top-down 701 and side view 702 of exemplary wound W). This emitter may move internally or externally to perform scanning, or emit diffusely. In some embodiments, the probe itself is subject to frameless stereotaxy to allow range measurements to be computationally combined while moving the probe within the wound. In some embodiments, single or multi-wavelength spectrometry and/or absorptiometry from the same emitter or emitters used for rangefinding, can be used to determine the tissue composition of the wound. In such embodiments, IS pharmaceutical doses (dose per cm2 WSA), can potentially be modified based on the fraction of certain tissue types present in the wound. Examples of tissue parameters assayed for include, but are not limited to, fractions of the WSA represented by muscle, adipose, bone, viscera, pleura, mesentery, vessels, neural (central or peripheral), meninges, enteric, tendon, ligament, and/or joint surfaces. Presence or absence of these tissues, and other conditions, may alter the rate of diffusion of a drug through the wound surface into the systemic circulation. Presence or absence of these tissues may present differing local toxicity or side-effect issues, which could justify modifying the delivered dose from the original calculated amount based on the WSA. In some embodiments, a lookup table is used to determine the total IS drug dose after a wound surface area is determined, to reduce error. In other embodiments, this calculation is performed automatically by a computer. Utilization of techniques like those depicted in FIG. 7 are can be used in instances of highly irregular wounds, such as those caused by trauma.

As previously stated, the IS pharmaceutical have one or more of the following characteristics: non-toxic or low toxicity to local tissues, even at high concentrations, absorbs slowly or not at all into systemic circulation, and is potent at treating its target disease state. When these conditions are met or nearly met, placement of the pharmaceutical directly into the wound accomplishes targeted drug delivery whereby intended local effects are enhanced and systemic side effects and toxicities are avoided. Additionally, when these conditions are met, the effective dose is relatively low while the toxic dose is relatively high, indicating the pharmaceutical will exhibit a high therapeutic index when delivered via the IS route of administration. Such instances (high therapeutic index in the context of targeted drug delivery), reduce safety concerns and reduce the requirement for strict dosing accuracy. In some circumstances, some IS pharmaceuticals may only need to be dosed approximately but high enough to assure no treatment failures. Such a condition may exist with vancomycin and some other known pharmaceuticals, as disclosed herein.

### 7) IS Pharmaceutical Administration Methods

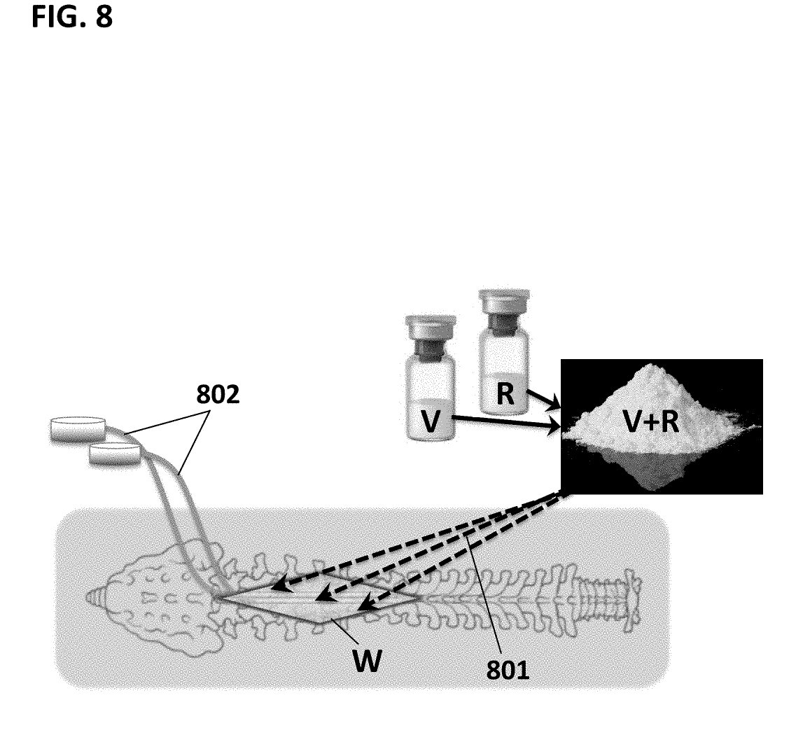

FIG. 8. depicts an exemplary schematic of basic manual IS administration of pharmaceuticals to a wound W. In this embodiment, an example is made of ultrapurified vancomycin V and rifaximin R, combined V+R at a specific ratio, which reflects one way of broadening the spectrum of antimicrobial activity for treatment and prevention of wound infections. This combination is schematized showing two bottles but this disclosure contemplates that the medications could be supplied pre-mixed or separately (so that dose ratios are not fixed/predetermined). The appropriate quantity of powdered or wetted drug is then applied (arrows 801), to the WSA deep to the epidermis. In some embodiments, part of the total dose is applied immediately after the wound is opened, and the remainder is applied at the completion of surgery or the completion of traumatic wound debridement. In some embodiments, part of the total dose is applied directly to surgical implants or incorporated into surgical graft tissue (bone, for example). In other embodiments, surgical implants or graft tissue are soaked in or rubbed with part of the pharmaceutical dose, prior to implantation.

In some instances, IS administration of suitable medications may increase seroma osmotic pressure if they are not broken down or absorbed through the wound. Under these circumstances, if wounds are fully closed, surgical drains or other means can be used to evacuate seroma fluid for several days after closure in order to avoid a higher risk of wound dehiscence. Therefore, in some embodiments, wounds are closed over a drain or drains 802, in order to allow for egress of non-absorbed drug as well as seroma fluid and blood. In other embodiments, wounds can be needle-tapped after closure or left open to heal by secondary intention, potentially with the aid of a negative pressure dressing.

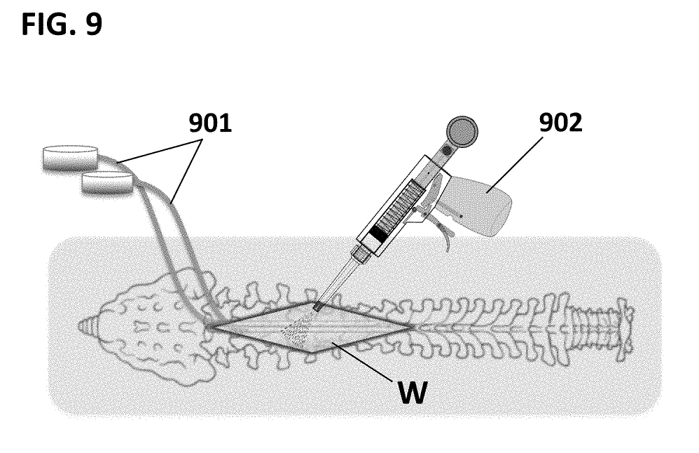

FIG. 9. depicts an alternate exemplary embodiment of the method of IS pharmaceutical administration utilizing a spray device 902 (designs disclosed in detail below). In this embodiment, IS medication dose is calculated based on measurement of WSA with any necessary adjustments for wound W tissue composition. The calculated dose is reconstituted into solution at a known concentration and loaded into the spray device 902 (methods detailed below). The spray device is then utilized to administer the correct dose of liquid phase IS medication to the surface area of the wound deep to the epidermis. Advantages of spray application by the devices disclosed herein include but are not limited to greater homogeneity of dose application, avoiding aerosolization of lyophilized medications, as well as greater assurance and ease of sterile drug delivery to the wound in both sterile operating theater and non-sterile field environments.

### 8) Additional Considerations for IS Administration

There are three main concerns with regard to the delivery of IS medications that are considered in the mode and method of drug delivery: 1) reliably delivering sterile drug to the wound without contamination (to avoid inoculating the wound during drug delivery); 2) avoiding aerosolization of drug and inhalation by practitioners; and 3) ensuring sufficiently directed and/or homogeneous application of drug to the wound surface area to improve distribution while avoiding detrimental dose concentration or dilution in certain areas of the wound. In some embodiments, the medication and any delivery devices or aids are packaged in a standard double-jacketed sterile fashion so that the outer packaging (which is non-sterile on the outside, sterile on the inside), is peeled away during delivery to the operating field, while the inner sterile sealed packaging is removed on the field, ensuring sterile delivery of the medication. In other embodiments, on-sterile-field methods of ensuring sterilization of drug are practiced, including but not limited to irradiation with UV light, heating, or dissolution in solvent which is toxic to microbes (examples may include alcohol, chlorhexidine solution, etc.). In other embodiments intended for use in the field, rather than in the operating theater, outer packaging is peeled away, revealing a spray device and other components (medication or solvent vials, propellant, etc.), which may be sterile inside and outside upon package opening, but are designed to be grasped/manipulated on their external surfaces by non-sterile hands. Despite this non-sterile handling, the design allows full operation of the spray device while maintaining the sterility of inner components and contents (drug, solvent, propellant, etc.), thereby facilitating sterile IS medication delivery to traumatic wounds by personnel in the field.

In some embodiments, avoiding aerosolization of drug and subsequent inhalation by practitioners is accomplished by the requirement to respirator dusk masks during drug delivery. In some circumstances, this is impractical and the placement/wearing of filter masks during surgery could contribute to sterile field contamination in some situations. In some embodiments, prevention of aerosolization of “dusty” lyophilized medications is achieved through wetting or dissolution. In some embodiments, slight wetting with an innocuous wetting agent like water or saline to make a paste is sufficient. In other embodiments, particularly with larger wounds, dissolution and delivery via a spray device is a more practical means of preventing aerosolization and improving homogeneity of delivery simultaneously. In these embodiments, it is contemplated that relatively low flow and low drive pressures as well as relatively larger nozzle diameters would be advantageous in preventing the formation of aerosolized droplets. In general, this would mean droplets not smaller than approximately 50 μm in diameter as droplets larger than this are unlikely to reach bronchiolar depths of the lung due to inertial impaction in the upper airway. Furthermore, droplets <50 μm in diameter fall out of suspension in air quickly and do not present high risk for inhalation. In some embodiments, a gelling agent, either of polysaccharide or protein-based chemistry may be added to the solvent at the time of spray application to aid in tissue adherence. However, delivery of some protein-bound drugs, like vancomycin, may be impaired by the use of proteinaceous gelling agents, making the drug less likely to bind to anchored proteins within the wound.

In some embodiments, non-aerosolization and homogeneity of application to surfaces is accomplished by application of the drug in the form of a sheet which covers the surface area of the wound. In these embodiments, cutting the sheet to fit the wound surface area accomplishes the measurement of wound surface area and the proper dosing simultaneously. In some embodiments, the drug is adhered to the surface of the sheet and then transferred to the wound surface area upon contact, with the sheet subsequently removed and discarded. In other embodiments, the drug is incorporated homogeneously into a dissolvable polymer sheet which dissolves and is broken down upon application to the wound surface area, transferring the drug to the wound surface in the process. These embodiments may employ a variety of possible polymers including but not limited to: microcrystalline cellulose, maltodextrin, and maltotriose, etc. These embodiments may employ a variety of possible plasticizing agents including but not limited to: glycerol, propylene glycol, polyethylene glycols, phthalate, and citrate derivatives.

As with any pharmaceutical delivered by any route of administration, efficacy and safety risks of IS medications are related to their dose-dependent effects on the target tissue and systemic off-target effects. The IS administration methods disclosed herein are methods of targeted drug delivery to a wound (either surgical or traumatic) because they concentrate drug at the site of disease (wound), and minimize drug concentration in off target areas. This is in contrast to the current prevailing methods of administration that rely on systemic distribution of a drug. This disclosure also provides methods for dosing medications administered IS based on the surface area of the wound. The primary volume of distribution for a drug administered by the IS methods of this disclosure is determined by the size of the wound for two primary reasons: 1) the “size” of target tissue to be treated by the drug is the internal surface area of the wound, 2) the rate of production of seroma fluid within a wound, which causes time-dependent dilution of an IS medication after application, is primarily determined by the internal surface area of the wound. Additionally, the risk of systemic toxicities and side-effects from a medication delivered by IS administration is primarily determined by the peak systemic drug concentration after a single IS dose to the wound. The rate of systemic diffusion of a medication delivered by IS administration, which dictates peak systemic drug concentrations after an IS dose, is primarily determined by two variables: 1) drug concentration within the wound, and 2) the surface area of contact for potential diffusion into the circulation. This surface area determines the initial volume of distribution and therefore dose-dependent drug concentration at the target site of disease, which is directly related to efficacy. The wound surface area also determines the dose-dependent rate of systemic diffusion and therefore is directly related to off target effects and systemic toxicities. Therefore, dosing of IS medications is calculated based on the surface area of the internal wound tissue. A third variable, and potential modifier of dosing parameters, may be the tissue type composition of a wound. For example, the fraction of the total wound surface area occupied by bone, muscle, fat, viscera, etc., may influence the potential for systemic diffusion as well as local or systemic off-target effects.

### 9) Devices for IS Administration of Pharmaceuticals

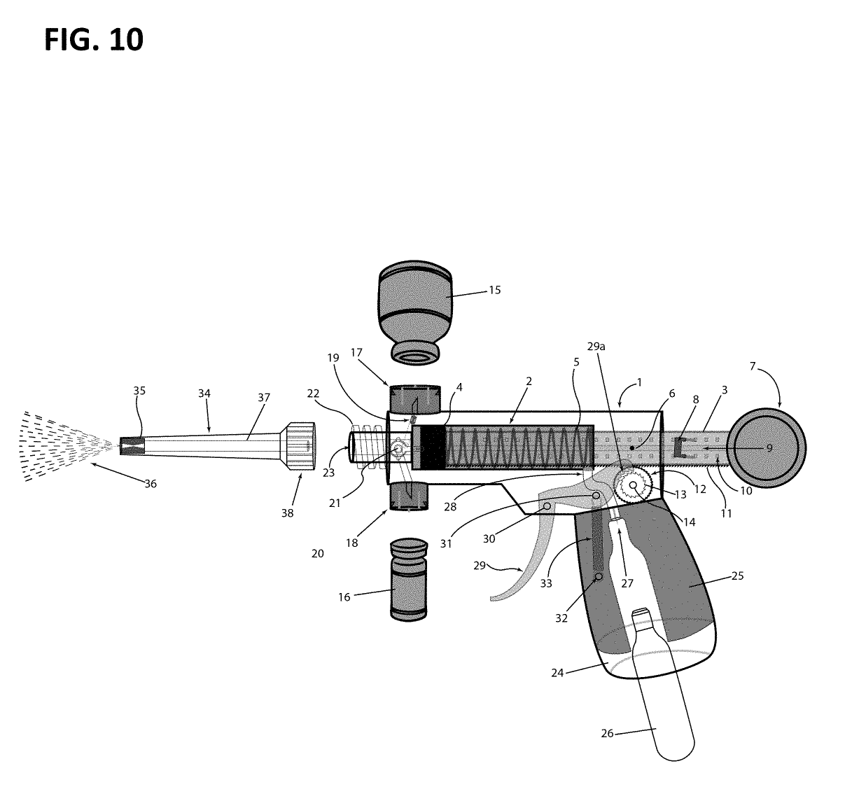

FIG. 10 depicts an exemplary embodiment of an intrasite medication spray applicator assembly comprised in its basic form of a receiver 1, a handle 24, a piston tube 2 installed inside the receiver, a male-threaded outlet fitting 22 mounted to the front of the receiver, a female-threaded spray nozzle tip 34 that may be screwed onto the male-threaded outlet fitting, a charging arm 3, and a ratchet-release trigger assembly 29.

In this embodiment, a vented snap-on vial access device 17 is mounted to the superior surface of the receiver 1. This vial access device 17 is of the correct diameter for attachment to a vial of sterile solvent 15 such as saline, water, Ringer's solution, etc. In some embodiments, the solvent vial 15 connects to this snap-on vial access device 17 in an inverted orientation to facilitate withdrawal of the solvent into the piston tube chamber 2. The vial access device is connected to a port in the outlet inner tube 23 between the front end of the piston 2 and the rear end of the threaded portion of the outlet 22 via a tube or conduit within the body of the receiver 1. This tube or conduit contains or includes a one-way flow check valve 19 to prevent reflux of fluid from the piston tube chamber 2 back into the solvent vial 15 after solvent is withdrawn from the vial 15 and into the piston tube chamber 2.

In this embodiment a second vented snap-on vial access device 18 is mounted to the inferior surface of the receiver 1. This vial access device 18 is of the correct diameter for attachment to a vial of sterile lyophilized medication 16. In some embodiments, the medication vial 16 connects to this snap-on vial access device 18 in a cap-upward vertical orientation to facilitate filling of the medication vial 16 with solvent from the piston chamber 2 via the vented snap-on vial access device 18. This vented vial access device 18 is connected to a stopcock 21 mounted through a port into the inner tube 23 of the outlet between the front end of the piston 2 and the rear end of the threaded portion of the outlet 22 via a tube or conduit within the body of the receiver 1. In this embodiment, the stopcock 21 is placed anterior to the port for the solvent tube.

The stopcock 21 is designed to allow switching of fluid flow direction between the piston 2, the medication vial 16, and the threaded outlet inner tube 23. In this depiction, positioning the “off” lever arm posteriorly, in the direction toward the piston chamber 2, results in the sprayer unable to fire and this provides a safety mechanism against accidental discharge. Positioning the “off” lever arm anteriorly, in the direction toward the front of the outlet 22, also prevents discharge but allows flow of fluid between the piston tube chamber 2 and the medication vial 16. In this embodiment, the flow of fluid while in this anterior stopcock position can occur either from the piston tube into the medication vial or in reverse. This allows lyophilized medication to be dissolved by solvent pushed into the vial 16 from the piston chamber 2. Dissolved medication can then be withdrawn from the vial 16 back into the piston tube chamber 2 without changing the stopcock position. In this embodiment, the intrasite spray applicator assembly can be rolled about its long horizontal axis to invert the medication vial 16 and facilitate withdrawal of dissolved medication into the piston chamber 2 via the vented vial access device 18. Positioning the “off” lever of the stopcock 21 inferiorly, in the direction of the medication vial 16, allows forward flow of fluid from the piston chamber 2 through the outlet's inner tube 23 when the trigger mechanism 29 is depressed.

In this embodiment, the piston tube comprises a tube chamber 2, a piston seal 4, and a charging arm 3. The piston seal may be made from a variety of materials. In some embodiments, chemically inert polysiloxane is used. In the embodiment of FIG. 10, forward force on the piston seal is provided by a compression coil spring 5 which has an outer diameter small enough to fit within the posterior piston chamber 2, and an inner diameter large enough to allow free longitudinal travel of the charging arm 3. An alternate or assistive means of piston drive force is also depicted in the form of a compressed gas canister 26 which can be inserted into an appropriately sized cutout within the handle 25 and engage with a press fit perforating connector 27. This perforating connector may be connected to the posterior piston chamber by means of a pressure tolerant tube or conduit 28. The compressed gas alternative can be used when greater piston drive pressure is desired, for example when delivering more viscous solutions through the spray applicator. The charging arm is equipped with a charging handle 7, for the purpose of a firm grasp during charging. Here the charging handle 7 is depicted as a ring, though other shapes are possible without substantially altering function.

Charging is accomplished by grasping the charging handle 7 and pulling posteriorly to compress the drive spring 5. Forward motion of the charging arm 3 and piston seal 4 (discharge of the spray applicator), is arrested by the engagement of ratchet teeth 11 on the inferior surface of the charging arm 3 with a ratchet gear 12 with the same sized teeth oriented in the reverse. This ratchet gear is held firmly in the optimal location for ratchet teeth engagement with the charging arm's ratchet teeth by means of a through-pin axle 14. The charging arm is held in optimal alignment for ratchet teeth engagement by means of a through guide pin 6, which prevents vertical flexibility of the charging arm from causing ratchet teeth disengagement. The charging arm 3 travels longitudinally over the through guide pin 6 by means of a longitudinal slot cutout 9 in the charging arm 3. In this embodiment, one means of dose limiting or dose dividing is depicted by a two-armed u-pin 8, which can be inserted through paired holes 10 in the charging arm 3. A sprayed dose of medication can be limited by placing the u-pin 8 through the paired holes 10 after charging. Discharge of the spray applicator is arrested when the u-pin 8 contacts the posterior surface of the receiver 1, thereby limiting the dose delivered to a fraction of the total within the piston chamber 2. The positions of the paired holes 10 on the charging arm may be inscribed with measurement numbers to assist in accurate dose delivery (not depicted). Alternatively, a sight window may be equipped on the side of the receiver for the purpose of allowing the user to see inscribed graduations on the piston tube chamber (not depicted). The overall operation of this embodiment is intended to be sufficiently similar to the operation of M16 variant military rifles, particularly with regard to handling during charging and deployment of the spray device, as to be familiar in function to a military medic or this like. The purpose of these design features, as well as the potential for larger-capacity drug delivery in this embodiment is to facilitate use by medical treatment personnel in battle-related casualty scenarios where high-stress and the need to treat multiple casualties simultaneously could impair the use of other devices.

In this embodiment the trigger mechanism 29 is comprised of a single machined piece which rotates around a through-pin 30 when the trigger lever is depressed. Trigger depression thereby raises the posterior portion of the trigger assembly, disengaging its ratchet engagement teeth 29a from the reverse ratchet gear 13. An extension spring 33 provides a constant downward force on the posterior trigger assembly, keeping its ratchet teeth engaged with the reverse ratchet gear 13. This extension spring pulls from a hole cutout in the posterior trigger lever 31 inferiorly to a through-pin in the handle 32. The reverse ratchet gear 13 is coaxial on the same axle through-pin 14, with the main ratchet gear 12, that engages with the charging arm ratchet teeth 11. The two ratchet gears 12 and 13 are fixed together and not allowed to rotated relative to each other. Therefore, when the trigger lever ratchet teeth 29a are engaged with the reverse ratchet gear 13, rotation of both ratchet gears 12&13 is halted, which in turn stops forward motion of the charging arm 3 and piston seal 4 via main ratchet gear 12 teeth engaging with charging arm teeth 11. This dual ratchet mechanism arrests discharge of the spray applicator until the trigger 29 is depressed and provides the means of cocking the spray applicator.

An example spray tip 34 is depicted comprised of a knurled base 38 with female threading to match the male threaded outlet 22, a shaft with an inner tube 37 designed to minimize dead space, and a Venturi flow restrictor 35 with a spray shaper nozzle tip (not depicted). The outer diameter of the inner tube 37 of the spray tip 34 is designed to have a tapered-contact water-tight fit into the inner diameter of the inner tube 23 of the male threaded outlet 22. The purpose of this feature is to prevent fluid leaks at the spray tip 34 attachment site (38 onto 22) during discharge of the intrasite spray applicator. The male and female threads of this attachment are designed as box threads to avoid cross-threading. A spray of intrasite medication solution 36 is depicted emanating from the spray tip nozzle 35.

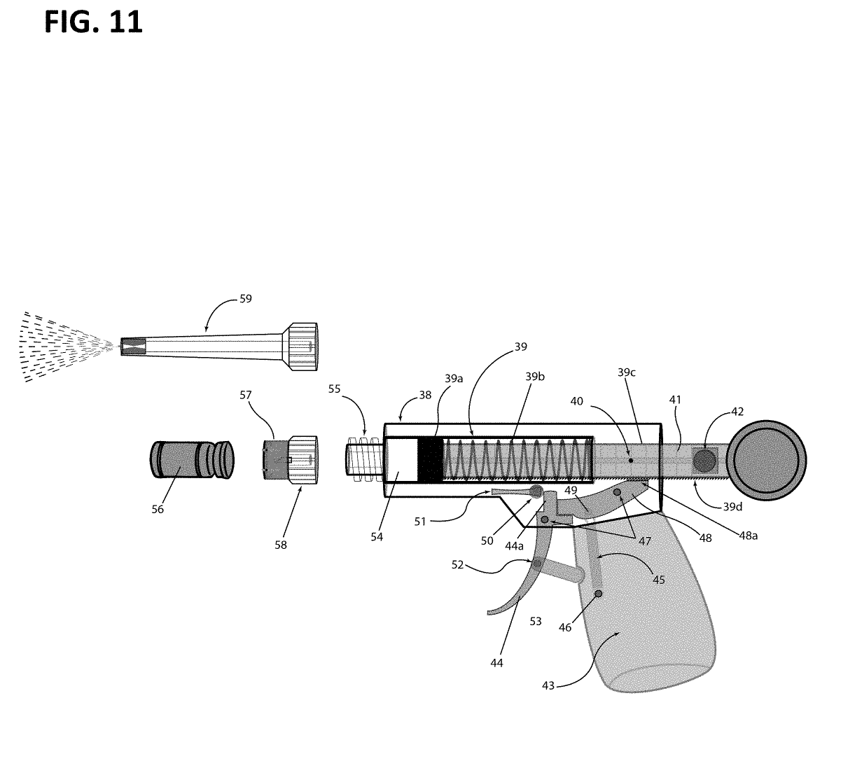

FIG. 11 depicts an alternate embodiment of the intrasite medication spray applicator assembly comprised in its basic form of a receiver 38, a handle 43, a piston tube 39 installed inside the receiver, a male-threaded outlet fitting 55 mounted to the front of the receiver, a female-threaded spray nozzle tip 59 that may be screwed onto the male-threaded outlet fitting, a charging arm 39c, and a ratchet-release trigger assembly 44.

In this embodiment a vented snap-on vial access device 57 is mounted to the female threaded knurled base 58 from other depictions of the spray tip 59. This vented vial access device 57 is of the correct diameter for attachment to a vial of sterile dissolved medication 56. In some applications this may be reconstituted lyophilized medication or, in other situations, medication that is stored in liquid form. In some embodiments, the liquid-filled medication vial 56 connects to this snap-on vented vial access device 57, which is then threaded onto the male-threaded outlet 55. The entire intrasite medication spray applicator assembly is then held vertically, so that the medication vial is inverted to gravity, in order to facilitate charging of the piston chamber 54 with fluid from the vial 56 via the snap-on vial access device 57. After charging of the piston chamber 54, the vial 56 and threaded snap-on vial access device 57 may be unscrewed, removed, and replaced with the spray tip 59 in preparation for discharge.

In FIG. 11, as in FIG. 0.10, charging is accomplished by pulling posteriorly on the charging arm 39c using the charging handle. This action compresses the piston spring 39b between the piston seal 39a and the posterior wall of the piston tube 39 and simultaneously draws fluid into the piston chamber 54 from the vial 56. Ratchet teeth 39d on the inferior surface of the charging arm 39c engage with oppositely oriented teeth 48a on the posterior lever arm 48 of the trigger assembly 44. The engagement of these ratchet teeth in the charged position provides the mechanism for cocking the intrasite spray applicator. Ratchet teeth 39d on the charging arm 39c are held in optimal position for engagement by means of a through guide pin 40. The charging arm 39c travels longitudinally over the guide pin 40 by means of a slot cutout as depicted and described in FIG. 10. As depicted here, the charging arm can be inscribed in graduation marks 41 to facilitate dose measurement. Dose limitation or dividing doses can be accomplished by a fully captured knurled knob on a bolt 42 threaded into a large nut on the opposite side of the charging arm 39c. This bolt assembly 42 could travel longitudinally along the slot cutout in the charging arm 39c and be tightened down at any position along the charging arm after charging. Discharge of the spray applicator is arrested when the large square nut 42 contacts the posterior surface of the receiver 38. This is an alternative to the u-pin mechanism 8 depicted in FIG. 7, though a variety of other mechanisms are contemplated that would have like function.

A variant trigger assembly is depicted comprised of a trigger 44 and a ratchet sear lever 48. Both components are held in position and pivot around through-pins 47. Static downward force is applied to the anterior part of the ratchet sear lever 48 by means of an extension spring 45 which pulls from a cutout hole 49 to a through-pin in the handle 46. This anterior downward force translates into a static upward force on the ratchet sear teeth 48a forcing engagement with the charging handle ratchet teeth 39d. The anterior part of the ratchet sear lever 48 is lifted by depressing the trigger 44, translating into downward disengagement of the ratchet sear teeth 48a from the charging handle ratchet teeth 39d, resulting in discharge of the intrasite spray applicator. In this design, when the trigger 44 is released, the ratchet teeth 48a&39d reengage and discharge is arrested.

Variant types of safety mechanisms are depicted. In one variant a trigger lockout strut safety 52 is depicted hinging on a pin in the trigger lever 44. This lockout strut 52 engages in a detent cutout in the handle when the safety is engaged. To disengage the safety, the lockout strut is rotated downward, out of the detent cutout, allowing the trigger to be depressed. In another variant a rotating trigger block safety 51 is depicted. A safety actuator lever 51 is coaxially through-pinned with a round bushing containing a cutout 50 on one side of the bushing. This cutout is just wider in dimension that the trigger lever post 44a. When the safety lever 51 is rotated to the forward “fire” position, the cutout 51 allows the trigger lever post 44a to pass through the bushing, thereby allowing the trigger to rotate and lift and disengage the sear lever 48 to discharge the spray applicator. When the safety actuator lever is rotated into the “safe” position, the non-cutout region of the round bushing 51 faces and contacts the trigger lever post 44a, thereby preventing anterior motion and blocking trigger function. In some embodiments, the safety mechanism is positioned inferior to the posterior edge of the sear lever 48 and used to lockout the sear (not depicted), rather than the trigger. In some embodiments utilizing materials with higher flexibility, like plastics, are used in order to prevent trigger pressure in a flexible system from overriding the safety.

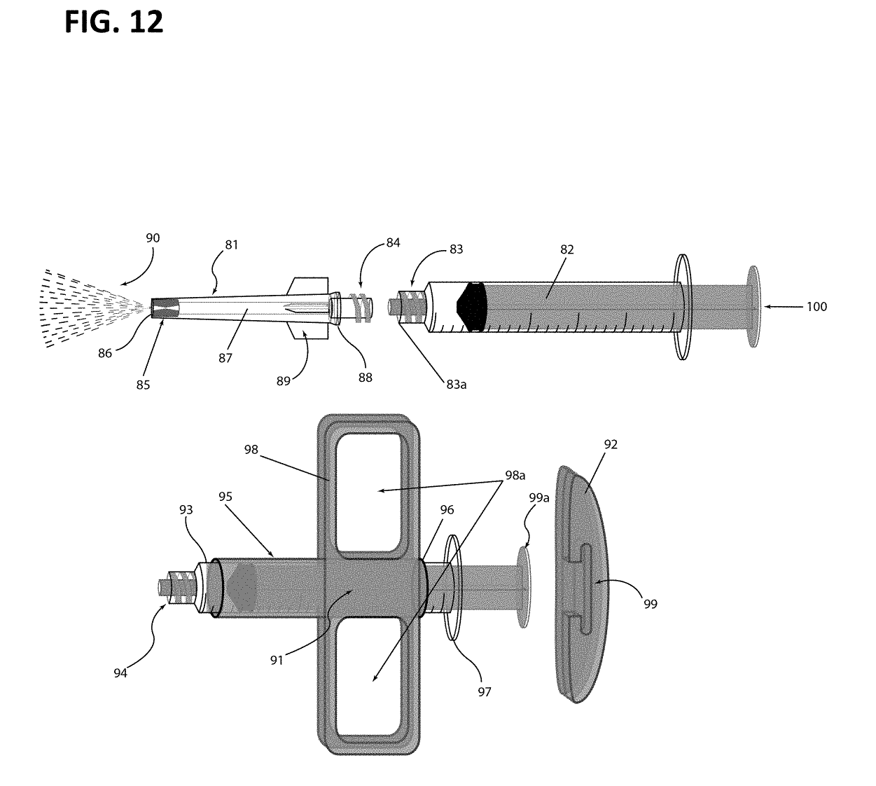

FIG. 12 depicts yet another alternate embodiment of the intrasite medication spray applicator. In the embodiment, the intrasite medical spray applicator is comprised of a spray nozzle tip 81 and a standard sterile syringe 82. The syringe 82 uses a male-threaded luer-lock outlet 83 and the spray nozzle tip 81 is modified from other forms depicted to attach onto the syringe utilizing a standard female-threaded luer-lock fitting 84. Utilization of this embodiment of the intrasite medication spray applicator device is accomplished by drawing liquid medication into the syringe 82 by standard means, attaching the spray tip applicator 81 by threading onto the syringe using the luer-lock fittings 83&84 until rotation is halted by the rotation stop collar flange 88 contacting the outer threaded tube of the male luer-lock fitting 83a on the syringe. This stop collar flange 88 is designed to lock the luer fitting threads by inducing strain to prevent unthreading during operation of the device. The user applies manual forward force to the syringe plunger 100 to create the drive pressure, forcing liquid medication from the syringe 82, into the spray nozzle tip inner lumen 87, through the Venturi restrictor 85, and out through the nozzle tip spray shaper 86, to create a medication spray 90. In this embodiment, firm tightening of the spray nozzle tip to the syringe is aided by the presence of fins 89 protruding from the sides of the rear portion of the shaft of the nozzle spray tip 81. Alternatively, this can be accomplished by other designs, including, but not limited to a knurled widened base of the spray nozzle tip as depicted in FIGS. 10-11, and FIG. 13.

A manual-drive assistance device 91 is also depicted which is comprised of a tube guide 95 for accepting a standard syringe fixated to a t-handle 98 with finger cutouts 98a, and a plunger base cap 92 with a cutout to accept the base of a syringe plunger. In this embodiment the syringe 93 is inserted into the guide-tube 95 until the base flange of the syringe 97 abuts the rear guide tube end 96. The plunger base flange 99a is then inserted into plunger base cap 92 via the cutout 99. The spray nozzle tip 81 is then attached to the syringe via the luer-lock fittings 84&94. In some variant embodiments, the syringe 93 can be fully captured within the guide-tube 95 by means of a threaded hand-tightened nut located on the outer diameter of the spray nozzle tip shaft (not depicted). As this nut is tightened, contact with the front guide-tube end would clamp the syringe 93 within the guide-tube 95. This manual drive assistance device is designed to provide the user with improved aiming and directability of the spray during application of intrasite medications as well as improved ability to apply consistent manual force without pain or injury to the fingers or palm of the hand.

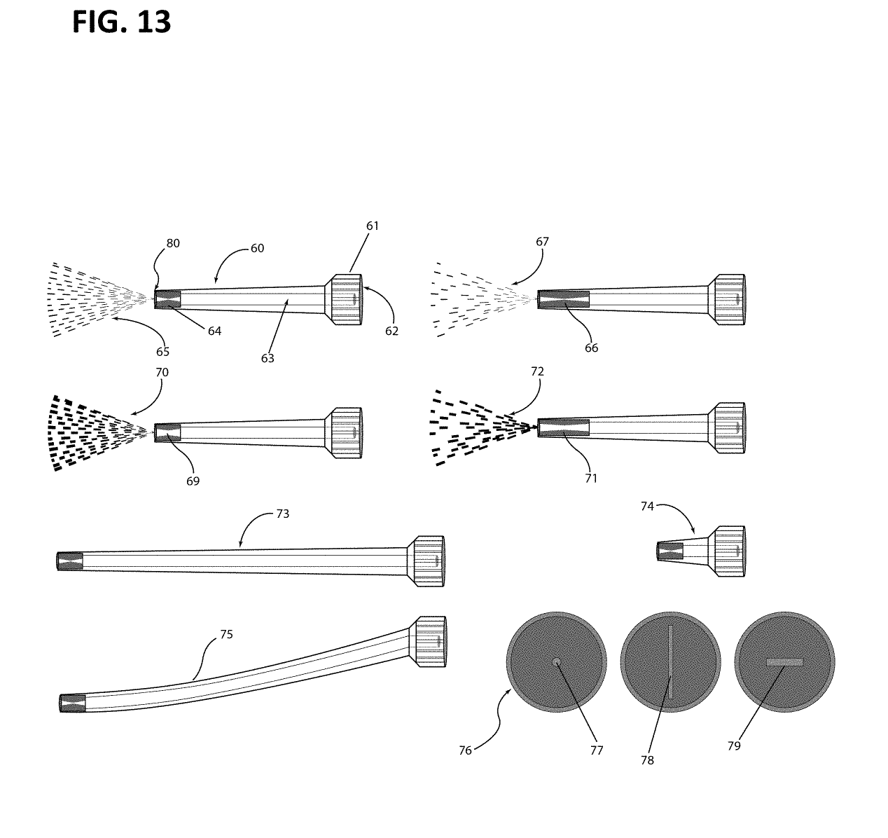

Referring to FIG. 13, exemplary spray tip variations and their features are depicted. There are many more varieties that are contemplated within the scope of this disclosure. The basic spray tip 60 (shown in side view) is comprised of a female internally-threaded 62, externally-knurled base 61, a shaft incorporating an inner tube or lumen 63, a Venturi restrictor 64 at the distal end, and a nozzle tip spray shaper 76 (shown in end-on view), which is located at the distal end 80 (shown in side view) of the spray tip assembly 60. Example spray 65 emanating from the nozzle end of the spray tip is depicted for orientation purposes and to demonstrate relative changes to spray qualities that are induced by changes to restrictor or nozzle tip design. Adjusting the length of the venture restrictor can alter the flow rate and therefore the volume of spray discharged as a function of time. A longer restrictor 66 can reduce the relative flow rate 67. A larger aperture in the restrictor 69 increases relative fluid flow rate and also increases droplet size 70. A longer restrictor with a larger aperture 71 may have mixed effects on spray qualities 72. Spray qualities are also dependent on drive pressure and fluid viscosity (not depicted). The optimal Venturi restrictor and nozzle tip spray shaper combination can be tuned to create a particular spray quality.

Spray tips may be equipped with long shafts 73 or short shafts 74. Long-shafted spray tips 73 contains more dead space but are useful for applying to distant surfaces for through minimal access corridors. Short-shafted spray tips 74 minimize dead space and improve spray device maneuverability. Spray tips may be built to be rigid 73, or flexible 75. Additionally, rigid but curved or angled spray tip designs may be advantageous in some circumstances or embodiments.

Several example nozzle tip spray shapers are depicted 76-79. These are positioned at the distal end of the spray tip 80, downstream of the Venturi restrictor 64. A circular hole in the nozzle tip 77 can generate a conical spray, while slotted (bar-shaped) nozzle tip holes 78&79 can generate varying orientations and morphologies of fan-shaped sprays. Differing spray shapes may be advantageous in different circumstances for assuring homogenous dosing of medication to the surface area of a wound. The combined effects of drive pressure, fluid viscosity, Venturi restrictor length and diameter, and nozzle tip aperture size and shape will determine the flow rate, dispersion, particle velocity and particle size emitted from the tip during discharge of the intrasite spray applicator. Many of these parameters will require optimization for specific circumstances, however, one consistent goal is to maintain droplet sizes larger than 100 μm to avoid aerosolization of medications and therefore prevent provider inhalation. Additionally, droplet velocities are calibrated to provide inertial impaction of droplets onto wound surfaces with minimal splash.

In the manually driven device depicted in FIG. 12, specific Venturi restrictor and nozzle tip spray shaper parameters depend on fluid viscosity but are also tuned to produce greater than 100 μm droplets at velocities that result in inertial impaction with minimum splash. It is estimated that maximum practical human grip strength is on the order of 600N. Drive pressures below this would result in larger droplets with less splash and still be considered safe from aerosolization.

### 10) Drugs Suitable for Intrasite Administration

Indications for and actions of IS drugs include, but are not limited to, antimicrobial (prevention, inhibition, or treatment of infections), antithrombotic, prothrombotic, antinecrotic, antiapoptotic, antineoplastic, chemotherapeutic, analgesic, antispasmodic, osteogenic, osteolytic activity, to prevent, inhibit, or promote wound healing, and/or function as growth factors or growth suppressors, among others. Medications which are known to be highly effective but have poor bioavailability represent favorable candidates for the IS administration methods of this disclosure. Medications with limited or absent local toxicity potential are also advantageous since high concentration single dose application is contemplated using the intrasite route of administration disclosed herein. Protein binding may be advantageous for IS drugs because this can act to anchor the drug within the wound and lead to a longer local half-life. In some embodiments, even drugs which some systemic toxicity potential that present risks for conventional IV or PO administration, can be safe and effective IS medications, so long as their rate of diffusion from the wound, into the circulation, is low. Given this, and the benefit of poor enteric absorption, the IS administration methods disclosed herein present an opportunity to take advantage of unused or underutilized medications to effectively treat patients. Furthermore, new classes of chemicals may become suitable IS medications that would otherwise be unacceptable due to safety or efficacy problems via current routes of administration.

IS therapeutic agents useful in the methods disclosed herein include low bioavailability agents whose concentrations in blood or internal tissues of a subject do not reach concentrations sufficient to produce an observable effect in the subject unless administered intravenously. Specific examples of medications that work well as IS drugs include vancomycin (and other glycopeptide antibiotics), rifaximin, tobramycin, antimicrobial peptides, thrombin, tranexamic acid, lidocaine, and amide local anesthetics. In some embodiments, these medications are administered in conjunction with one another. In some embodiments, vancomycin and rifaximin are administered together via the IS administration methods disclosed herein. Vancomycin and rifaximin have advantageous chemistry for IS administration and function in complementary and synergistic ways to prevent and treat a broad range of microbial infections, including gram-positive organisms, gram-negative organisms, anaerobes, biofilm forming organisms, and drug resistant organisms.

Vancomycin binds easily to proteins and has poor bioavailability. As a result, narrow dosing parameters are required to effectively treat infections with IV vancomycin, while maintaining an acceptably low risk of renal and other toxicities. On the other hand, vancomycin is nearly inabsorbable through the gut and PO forms of the drug have been effective at treating certain intestinal infections with nearly zero risk of systemic toxicity. Vancomycin is especially useful as an IS medication since it is not detectably absorbed into the blood stream through wound tissues. Similarly, rifaximin, a chemical variant of Rifamycin, which is highly active against most gram-negative organisms, demonstrates extremely poor absorption through the alimentary system and has been used to treat intestinal infections with low risk of systemic toxicity. It has been surprisingly discovered that Rifaximin is an ideal agent to combine with vancomycin to create a broad-spectrum antimicrobial drug specifically for the IS administration methods disclosed herein.

Formula I is the chemical structure of vancomycin.

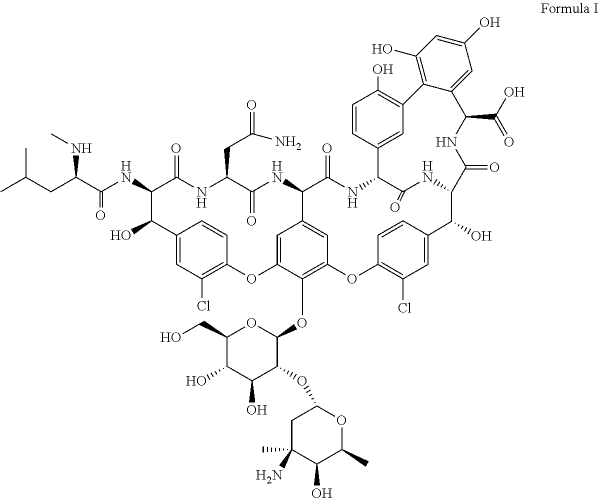 [SMILES: CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O)c3ccc(c(Cl)c3)Oc3cc2cc(c3O[C@@H]2O[C@H](CO)[C@@H](O)[C@H](O)[C@H]2O[C@H]2C[C@](C)(N)[C@H](O)[C@H](C)O2)Oc2ccc(cc2Cl)[C@H]1O]

Due to its large size and numerous benzene rings, vancomycin is not easily absorbed into the blood stream via the enteral system or absorbed through the tissues of a wound (either surgical or traumatic). Preliminary evidence (disclosed herein) shows that when vancomycin is applied directly to spinal surgery wound tissues in moderate doses (within the dosing guidelines for IV use), its concentration remains undetectable in the blood stream. Vancomycin has a relatively high affinity for proteins, both soluble albumen and anchored skeletal proteins, causing it to “stick” to the tissues of the wound. This property increases the washout time of vancomycin from a wound after it is applied. Preliminary evidence indicates that when used in moderate doses, vancomycin remains in high enough concentration for efficient suppression of gram-positive microorganisms for at least 4 days. This remains true even when accumulating seroma fluid is removed from the wound via drains. The lack of diffusion through the wound into the circulation as well as its affinity for anchored proteins, make vancomycin ideally suited for use as an IS antimicrobial. Compared to the IV route of administration, when vancomycin is applied IS, these chemical properties improve safety by reducing systemic toxicity risk and improve antimicrobial efficacy by increasing drug concentrations at the site of potential infection.

Formula II is the chemical structure of Rifaximin.

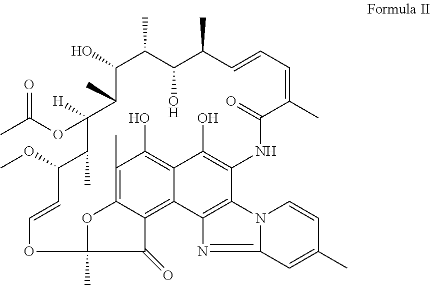

Rifaximin is a semisynthetic antibiotic based on rifamycin, which has very poor oral bioavailability (<0.4%) due to its additional pyridoimidazole ring. Rifaximin binds to bacterial DNA-dependent RNA polymerase and prevents catalysis of polymerization of base-units onto a DNA strand, inhibiting bacterial RNA synthesis. Rifaximin has broad-spectrum antimicrobial activity against aerobic and anaerobic gram-negative and gram-positive organisms, and is effective against biofilms. It is moderately protein bound, and non-toxic to mammalian cells. Stimulation of resistance is known to be less than that observed with rifamycin treatment and is non-plasmid based. All of these chemical and antimicrobial properties make rifaximin suitable for use in the IS administration methods of this disclosure. It is also ideally suited to pair with vancomycin since its spectrum of coverage includes anaerobic and gram-negative organisms (for which vancomycin has very poor activity). Rifaximin is also particularly useful in IS administration to surgical implants due to its activity against biofilms. As with vancomycin, there are significant safety and efficacy advantages of IS rifaximin when compared to standard IV antimicrobials.

Formula III is the chemical structure of tobramycin.

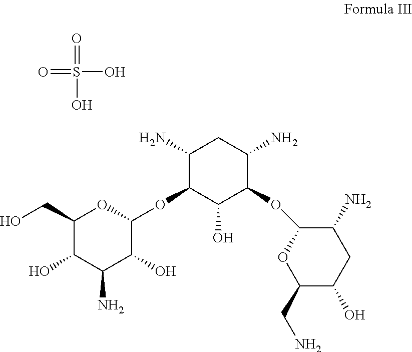 [SMILES: O=S(=O)(O)O ; NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](N)C[C@@H]1O]

Tobramycin is an aminoglycoside antibiotic with molecular mass 0.47 kDa produced by streptomyces and administered IV to treat primarily gram-negative bacterial infections. It is particularly useful against difficult to eradicate pseudomonas. Currently, tobramycin is only used IV due to very poor oral bioavailability. Tobramycin works by binding to bacterial 30S and 50S ribosome subunits, preventing mRNA from being translated into protein, resulting in cell death. Like other aminoglycosides, tobramycin is ototoxic and nephrotoxic when administered IV. This is particularly true when multiple IV doses accumulate over time or kidney filtration rate declines. Due to this, tobramycin has a narrow therapeutic index when administered IV. On the other hand, with single time dosing, the potential for poor diffusion in to the circulation, and excellent activity against hard to treat gram-negative microbes, tobramycin or tobramycin conjugates can be used in the IS administration methods of this disclosure.

Formula IV is the chemical structure of Amphotericin B.

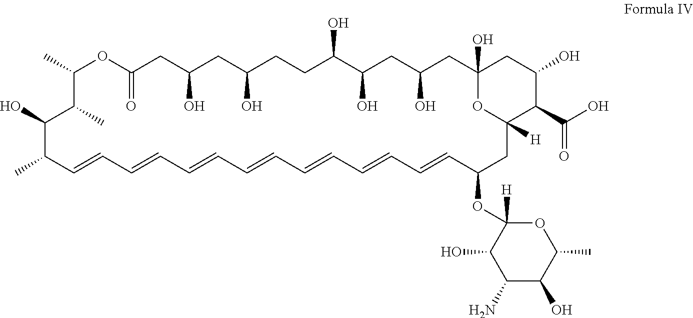 [SMILES: C[C@@H]1[C@H](O)[C@@H](C)/C=C/C=C/C=C/C=C/C=C/C=C/C=C/[C@H](O[C@@H]2O[C@H](C)[C@@H](O)[C@H](N)[C@@H]2O)C[C@@H]2O[C@](O)(C[C@@H](O)C[C@@H](O)[C@H](O)CC[C@@H](O)C[C@@H](O)CC(=O)O[C@H]1C)C[C@H](O)[C@H]2C(=O)O]

Amphotericin B is an amphoteric, potent, broad-spectrum antifungal polyene chemical of molecular mass 0.92 kDa. Amphotericin B is fungicidal against a variety of Aspergillus, Candida, Cryptococcus, and Fusarium species, among others, that cause invasive wound-related infections. It exhibits very low oral bioavailability, is highly protein-hound, and exhibits relatively poor tissue penetration when systemically administered. Additionally, its antifungal potency is not diminished by the presence of blood serum or serum proteins. These properties make amphotericin B an excellent candidate IS antifungal agent. While traditional IV infusion of this drug has been complicated by significant systemic toxicities and reactions possibly related to wide-spread histamine liberation, these problems may be avoided by IS administration since the rate of diffusion from the wound into the circulation is low. Amphotericin B is known to have effects on mammalian cell membranes at high concentrations. Thus, IS dosing of amphotericin B require greater accuracy than some other IS medications. To address this issue, in some embodiments, conjugation or IS administration in drug loaded hydrogel form, as disclosed herein, can be employed with amphotericin B to allow a flatter drug concentration curve over time.

Formula is the chemical structure of the two enantiomeric forms of itraconazole.

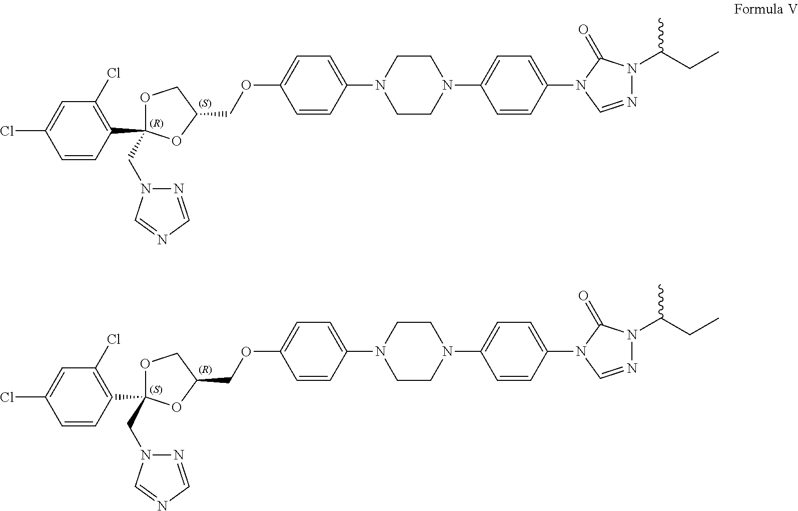

Itraconazole is a large lipophilic azole antifungal of molecular mass 0.705 kDa which is highly protein-bound and exhibits relatively poor oral bioavailability and tissue penetration when administered systemically. It has broad spectrum activity against a variety of species that cause invasive fungal wound infections including relatives of Aspergillus, Mucorales, Fusarium, Scedosporium, Blastomycosis, Sporotrichosis, Histoplasmosis, Candida, Cryptococcus, and others. For these reasons, Itraconazole is an excellent candidate IS antifungal agent, particularly when dealing with blast-related war wounds. Many of the same concerns and strategies for addressing them that exist for Amphotericin B, also apply to Itraconazole.

Thrombin is a globular serine protease enzyme with molecular mass 36 kDa. Thrombin converts soluble fibrinogen into insoluble strands of fibrin, as well as catalyzing conversion of factors XI to XIa, VIII to VIIIa, V to Va, and XIII to XIIIa. Thrombin also promotes platelet activation and aggregation by activation of protease-activated receptors on platelet membranes. Thrombin is inactivated by endogenous antithrombin. Thrombin has found numerous uses in medicine and is currently employed in small doses, combined with carrier agents and approved as a device, to limit blood loss in surgical fields. It does not absorb quickly from the wound into the circulation due to its large size and rapid inactivation, and is unlikely to cause local toxicities. For these reasons, thrombin is an excellent candidate IS pharmaceutical.

Formula VI is the chemical structure of Thrombin.

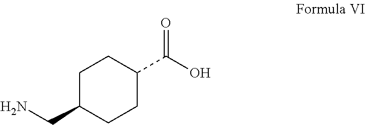 [SMILES: NC[C@H]1CC[C@H](C(=O)O)CC1]

Tranexamic Acid (TXA, trans-4-[aminomethyl]cyclohexanecarboxylic acid) is a synthetic analog of the amino acid lysine with molecular mass 0.157 kDa. It is a competitive inhibitor of plasminogen and a noncompetitive inhibitor of plasmin, which prevents plasmin/antiplasmin from binding to and degrading fibrin matrix structure. This action gently but effectively prevents clot breakdown. TXA has both oral and IV formulations, exhibits 34% oral bioavailability, has a high therapeutic index, is non-toxic to local tissues, has a half-life in the blood stream of approximately 2 hrs, and is generally considered safe.

Trauma causes inactivation of plasminogen activator-1, promoting fibrinolysis that is one cause of acute trauma-related coagulopathy. IV TXA reduces hemorrhage and all-cause mortality in major trauma, which is likely related to this inhibitory effect on trauma-related coagulopathy. IV TXA requires IV-access, which can be delayed until arrival at a medical center. Intrasite TXA administration to open traumatic wounds in the field may offer significant benefits of earlier treatment leading to less blood loss. Furthermore, IS TXA may allow more potent local effects to arrest wound hemorrhage than can occur through circulatory distribution from oral or IV administration of the drug. Additionally, with IS TXA there may be a benefit to diffusion through the wound into the circulation by reducing ongoing systemic coagulopathy.

Clot stability is required for timely wound healing and IS TXA may aid wound healing for both surgical and traumatic wounds through improved clot stability. Similarly, IS TXA may help prevent hematoma accumulation after wound closure, which is a cause of pain, secondary injury, and further medical and surgical interventions. IS delivery of TXA to wound tissues may present a safe and effective means of limiting blood loss and hematoma formation in surgical and traumatic wounds.

Formula VII is the chemical structure of Lidocaine.

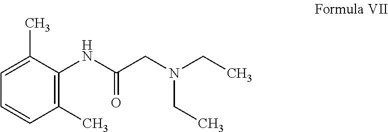 [SMILES: CCN(CC)CC(=O)Nc1c(C)cccc1C]

Amide-type anesthetics, such as lidocaine and bupivicaine, possess local anesthetic properties and cardiac stabilizing properties. Lidocaine is a mixed action drug of molecular mass 0.234 kDa with a primary action of halting signal conduction in neurons occurs by blocking fast voltage-gated Na+ channels in the neuronal cell membrane responsible for signal propagation. The same mechanism is responsible lidocaine's cardiac effects. While oral bioavailability is 35%, topical bioavailability is approximately 3%. Lidocaine is highly protein bound in circulation and is primarily metabolized in the liver and there are several active metabolites. Lidocaine is considered safe to use and with a high therapeutic index when administered via oral, IV, or topical routes.

Amide-type anesthetics are attractive IS medications for two reasons. One is the local inhibition of nociceptive pain as a method of pain control after surgery or trauma. There are acute and chronic complications related to opioid medications and a non-opioid adjunct for acute pain control is needed. Second, lidocaine directly modulates the innate immune system via actions on macrophages, monocytes, and polymorph neutrophils. Lidocaine may inhibit macrophage cell adhesion, chemotaxis, and phagocytosis as well as modulate reactive oxygen species creation. Polymorph neutrophil recruitment is reduced by lidocaine. Modulation of these innate immune system components may reduce inflammation at the site of injury and systemically by modulating chemokine expression by these cell types. Thus, an amide-anesthetic delivered IS, such as lidocaine, may have effects at the wound to decrease pain and reduce inflammation, further reducing the damage caused by a post-surgical or post-traumatic state. These anti-inflammatory actions can act synergistically with IS antimicrobials to reduce the incidence of wound related infections. Other, longer acting, members of the lidocaine family (bupivacaine, et al.), are also useful for IS treatment of wound pain, although these other drugs may or may not display the inflammatory cascade modulation of lidocaine.

Antimicrobial Peptides (also known as Host Defense Peptides or HDPs) are small endogenous peptide molecules found in all animals that are part of innate mechanisms of immune response to pathogens. These peptides occur in multiple genetic/morphological families and display potent antimicrobial activity against gram-negative and gram-positive bacteria, as well as fungi, and some viruses. Many antimicrobial peptides are immunomodulators. Additionally, some of these peptides have been shown promote growth of fibroblasts and keratinocytes and could potentially play a role in promoting wound healing. Different families of these peptides exhibit different mechanisms of action (both against pathogens and as endogenous modulators). Most antimicrobial peptides are amphipathic and usually between 12 and 50 amino acids long (10-50 kDa). Further, they have limited potential as IV or oral medications due to rapid inactivation and/or breakdown in these environments. However, these molecules or their conjugates are excellent candidates for use as IS pharmaceuticals.

### EXAMPLES

### Example 1

Intrasite Vancomycin Pharmacodynamics Trial.

A single dose cohort pharmacodynamics trial was performed under FDA IND#117494, the first FDA IND awarded for intrasite vancomycin. This dose cohort involved administration of a low dose of intrasite lyophilized vancomycin into complex instrumented spinal surgery wounds in adult patients. At multiple specific time points after surgery measurements were taken of vancomycin and endotoxin levels in the blood stream and from the wound seroma fluid (via the wound drain). Measurements of endotoxin levels within the blood stream and seroma fluid were also taken at the same time points following surgery. All personnel present during administration of IS vancomycin were required to wear a certified fit N-90 mask to prevent inhalation of aerosolized vancomycin powder, which is a known safety risk for pulmonary fibrosis.

Intrasite Dosing of Vancomycin.

Intrasite dosing was based on the surface area of the wound (tissues within the spinal wound lying deep to the skin). Wound surface area was estimated by a wound Length×Depth×2 calculation where length and mean depth were measured directed by the surgeon after surgical opening of the wound was completed. To assure safety, the maximum IS dose of vancomycin given to patients in this cohort was limited to well below the normal maximum safe IV dose of 2.5 g. Given that maximum wound surface areas created in spinal deformity patients are on the order of 1000 cm², a surface area dose of 2 mg/cm² provides for a maximum single dose of ˜2 g IS vancomycin, considered safe even if the entire dose absorbed into the circulation. To improve the homogeneity of IS dose application one third of the total calculated dose was delivered to the subepidermal tissues of the wound, superficial to the perimuscular fascia, one third of the total dose was applied to the subfascial muscle, bone, and other tissues, and one third of the total dose was milled with the bone graft before implantation.

Wound Vancomycin Levels Following a Single Intrasite Dose.

Wound seroma vancomycin levels were measured via the wound drain at multiple time points for 4 days after a single intrasite dose given at the conclusion of surgery. A surface area-based dose of 2 mg/cm² resulted in persistent vancomycin levels within both the subfascial (deep), and suprafascial (shallow), wound compartments sufficient to kill gram-positive organisms with any sensitivity to vancomycin, even some strains of Vancomycin-Resistant Staphylococcus aureus (VRSA). However, vancomycin is a time-dependent killer of microbes and levels must exceed kill concentrations for up to 48 hrs to be completely effective. Unfortunately, this level was not consistently exceeded at the 2 mg/cm² dose. Additionally, while mean drug levels were within the effective range for more than 48 hrs after administration within the suprafascial wound compartment, in some patients' individual data points were measured significantly below this threshold, arguing for an increased dose per unit surface area.

Suprafascial (Shallow) Wound Vancomycin Concentrations after IS Dose.

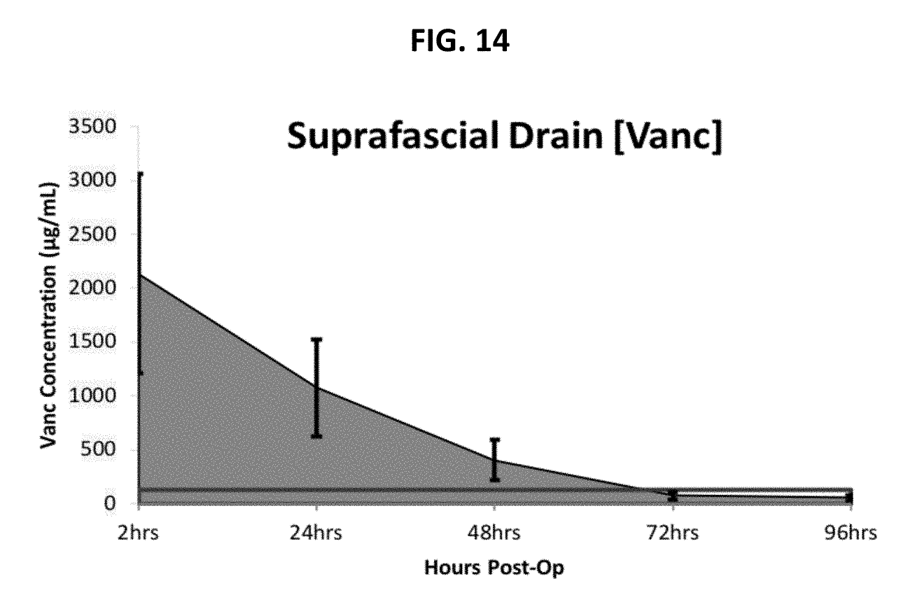

In the 10 patients of the 2 mg/cm² dose cohort mean vancomycin concentrations in the suprafascial wound compartment were found to significantly exceed threshold to kill VRSA for more than 48 hrs following a single IS dose. Straight red line represents two times the minimum 90% bactericidal concentration (MBC⁹⁰), for certain strains of VRSA (64 μg/ml×2=128 μg/ml), which is held to be a reliable kill concentration for microorganisms with any sensitivity to vancomycin. While the highest concentration measured at 2 hrs was 6110 μg/ml, the lowest concentration within the first 48 hrs was 16.2 μg/ml, well below this kill threshold for VRSA, indicating the potential for a treatment failure due to low dosing. Results are shown in FIG. 14.

Subfascial (Deep) Wound Vancomycin Concentrations after IS Dose.

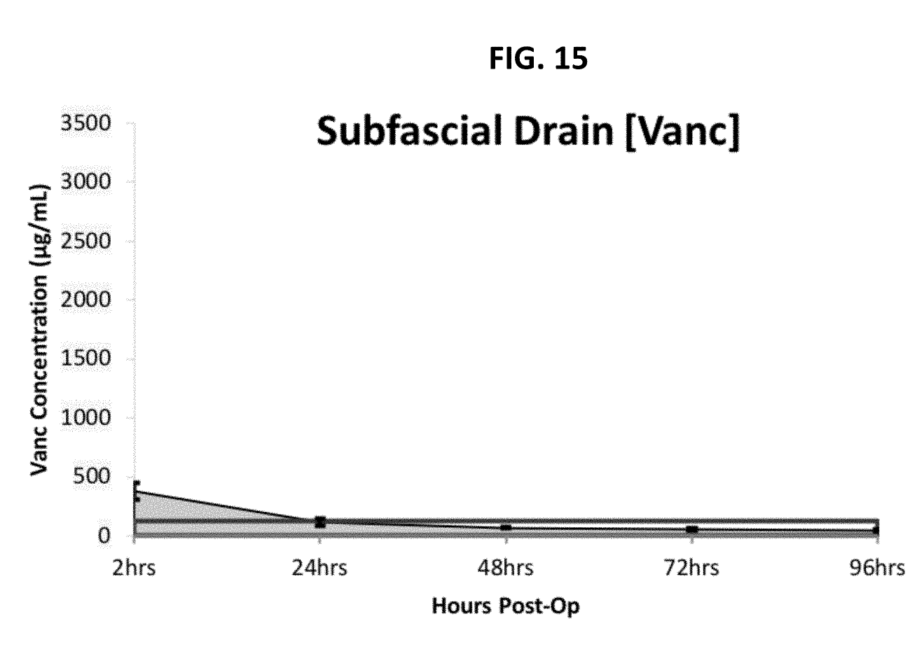

In the 10 patients of the 2 mg/cm² dose cohort mean vancomycin concentrations in the subfascial wound compartment were found to significantly exceed threshold concentration to kill VRSA for less than 24 hrs following a single dose. Straight red line represents two times the MBC⁹⁰ for VRSA (128 μg/ml), held to be a reliable kill concentration. Vancomycin in known to be a time-dependent killer of microorganisms that can take up to 48 hrs of consistently higher-than-threshold concentration to be bactericidal. Additionally, while the highest concentration measured at 2 hrs was 952 μg/ml, the lowest concentration within the first 48 hrs was 4.1 μg/ml, well below the reliable kill threshold for low-vancomycin-sensitivity microorganisms and indicating a significant potential for treatment failure due to low dosing. Results are shown in FIG. 15.

Systemic Circulation Serum Vancomycin Concentrations after IS Dose.

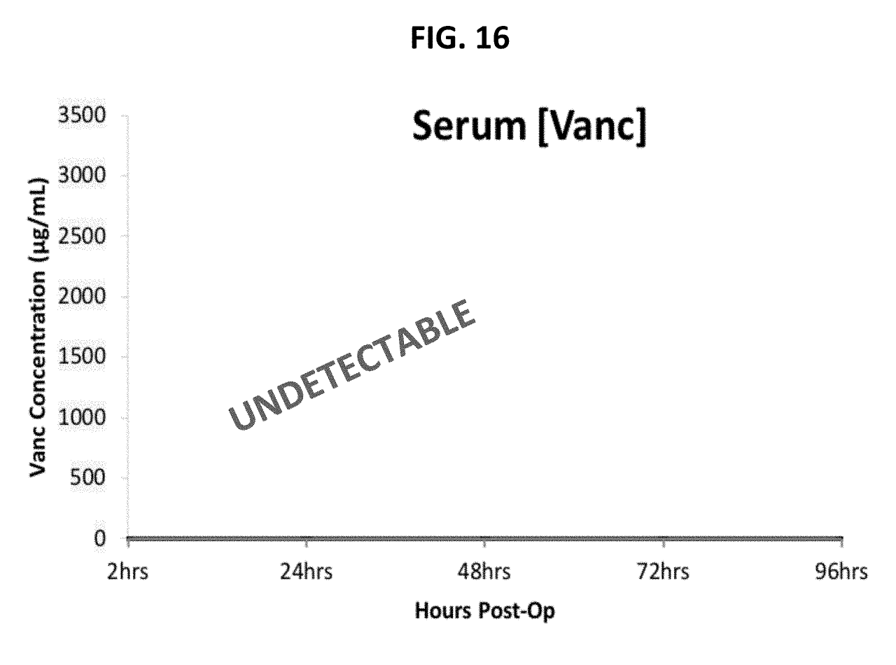

In all of the 10 patients of the 2 mg/cm² dose cohort serum vancomycin levels remained undetectable at all measured time-points after a single IS dose. Detectability limit in these experiments was >1.7 μg/ml. This is interpreted as a strong indicator of safety with regard to systemic side effects and toxicities as well as evidence of a very wide therapeutic index when vancomycin is administered IS. This strongly contrasts to the higher probability of systemic toxicity and narrow therapeutic index when vancomycin is administered IV. Results are shown in FIG. 16.

Systemic Circulation Serum Endotoxin Concentrations after IS Dose.

Serum endotoxin levels were measured at multiple time points after administration of a single IS vancomycin dose of 2 mg/cm². While examples of measurement difficulties and data censoring (indicated by <X values), were relatively common due to sample coagulation, multiple reliable measurements were made of serum endotoxin levels following IS vancomycin dosing. In multiple patients, at time points extending up to 96 hrs after surgery, endotoxin concentrations were measured at between 1-2 EU/mL. This concentration is well below the standard 5 EU/mL threshold commonly held to be the minimum concentration for inducing symptoms of endotoxemia in humans.

Systemic Circulation Serum Endotoxin Concentrations (EU/mL) at Five Time Points Following IS Dose of vancomycin.

Systemic circulation serum endotoxin concentrations were measured in 10 patients at 5 time intervals following IS administration of vancomycin. Results are shown in Table. 1

TABLE 1

| Pt | 2 hrs | 24 hrs | 48 hrs | 72 hrs | 96 hrs |
|---|---|---|---|---|---|
| 01 | 1.01 | <0.50 | <0.50 | <0.50 | <0.50 |
| 02 | <0.50 | <0.50 | <0.50 | <0.50 | <0.50 |
| 03 | error | 0.58 | <0.50 | 0.71 | 1.93 |
| 04 | 0.60 | <0.50 | <0.50 | <0.50 | <0.50 |
| 05 | 1.42 | <0.50 | <0.50 | <1.0 | <0.50 |
| 06 | 1.2 | 0.13 | <1.0 | 1.56 | <1.0 |
| 07 | <1.0 | <1.0 | <1.0 | <1.0 | 1.03 |
| 08 | <1.0 | <1.0 | <1.0 | <1.0 | <1.0 |
| 09 | <10.0 | <1.0 | <1.0 | <1.0 | <1.0 |
| 10 | <10.0 | <1.0 | <1.0 | <1.0 | <1.0 |

Adverse Reactions to IS Vancomycin.

In this small pharmacodynamics trial there were no patient- or practitioner-identified adverse reactions related to IS vancomycin. There was one example of a post-operative gram-negative bacterial surgical site infection which could not have been prevented by vancomycin because its spectrum of antimicrobial effects dies not cover this organism.

Dosing of IS Vancomycin.

While wound concentrations in the subfascial and suprafascial compartments after single 2 mg/cm2 IS doses of standard lyophilized vancomycin averaged well above the reliable kill threshold for VRSA, there were examples of recorded levels well below this threshold. Low measurements of this kind and frequency could lead to treatment failures, particular when treating, rather than preventing, surgical site infection. This fact argues for increasing doses of IS vancomycin from 2 mg/cm² that was tested in the forgoing experiments. Additionally, the wide concentration difference between the suprafascial and subfascial compartments argues for devices and/or methods for optimizing/homogenizing dose placement and distribution throughout the wound (addressed in this disclosure).

Endotoxemia Concerns from IS Vancomycin.

While challenges exist to the measurement of endotoxins in wound seroma fluid and blood serum, multiple reliable measurements of endotoxin levels between 1-2 EU/mL were made during this trial cohort. While these levels are well below the 5 EU/ml usually thought to be the threshold for symptoms of endotoxemia in humans, increasing the dose of IS vancomycin to combat potential treatment failures from low dosing (as described in the previous section) may lead to unacceptably high levels of endotoxin in the systemic circulation. This fact argues for using ultrapurification of vancomycin to remove endotoxins before the dose escalation trial can continue.

Application of Experimental Design.

Experiments are described for accomplishing the ultrapurification of IS vancomycin and IS vancomycin-rifaximin combination drug. These medications are used as specific examples but the experiments described may be applied to the removal of endotoxins and the FDA approval process for IS medications in general (though adjustments from this description may be required in some instances).

### Example 2

Endotoxin Removal from Vancomycin and/or Other IS Pharmaceuticals by Ultrapurification.

A variety of purification methods can be applied singly or in series to achieve the desired ˜10-fold reduction in endotoxin levels found in current formulations of vancomycin, in order to make the drug suitable for IS administration. Various embodiments of these methods are described herein. In order to achieve industrial scale production of endotoxin-removed vancomycin, vancomycin-rifaximin, or any other IS medication requiring this process, a series of cost-efficiency and scalability experiments is performed to determine which process or combination of processes are best suited to industrial/commercial scale production of that particular pharmaceutical. This scale up process uses sublot confirmatory batch testing for endotoxin levels and other impurities using methods disclosed herein. Methods for commercial scale production of endotoxin-removed vancomycin, vancomycin-rifaximin, or other pharmaceuticals, may change over time, due to economic factors, cost, efficiency, or availability changes of products, materials, or components of the process. Recrystalization and distillation are considered to be two commonly used options for commercial purification processes, though endotoxin removal by surface contact to polystyrene bound polymyxin B, as described herein, may prove to be even more efficient at industrial scales.

### Example 3

Testing Endotoxin Levels in Ultrapurified Pharmaceuticals.

A number of methods exist for quantitatively measuring the presence of endotoxins. The most commonly used is the Quantitative Limulus Amebocyte Lysate (qLAL) test, though newer and possibly more accurate and precise methods exist including Gas Chromatography/Mass Spectrometry (GC/MS), High Pressure Liquid Chromatography/Tandem Mass Spectrometry (HPLC/MS/MS), as well as Human Endothelial Cell E-selectin Binding assay. These methods are compared in our pharmaceutical samples (post purification), against standard positive and negative controls for accuracy, precision, and the reliability of measurements to detect endotoxin concentrations down to 0.01 EU/mL. The method capable of accurate and precise measurements down to 0.01 EU/mL which demonstrates highest cost efficiency is utilized for future testing of endotoxin levels on our pharmaceutical samples.

### Example 4

Pharmacodynamics and Safety Experiments in Animals.

Because IS administration is a new route of administration for pharmaceuticals a variety of pharmacodynamics experiments in animals are undertaken prior to human testing. Results of vancomycin testing are disclosed herein. For other candidate IS pharmaceuticals, pharmacodynamics experiments in animals use 10-50 animals. Model wounds are made surgically in a reproducible and standardized way which expose the tissues intended to be targeted by that medication. Escalating doses of IS medication are delivered using the methods disclosed herein and medication levels within the wound and the systemic circulation are tested at multiple regular intervals after surgery. These testing intervals vary depending on the needed half-life and action-time of that particular medication. Animals are monitored for side effects and toxicities using standardized blood labs and veterinary examinations. These experiments allow determination of preliminary safety as well as dose-selection for efficacy experiments in animals. In some instances, the most suitable model organism could be rodents or rabbits, while in other instances pigs are used because they more closely approximate the human body mass to surface area ratio, circulating volume, and soft tissue composition. In some instances, experiments in multiple animal models are conducted before proceeding to human trials. In some instances, multiple sizes and types of wounds are tested to understand how the pharmacodynamics, side effects, and toxicities may be affected by contact with different types and surface areas of subepidermal tissues (muscle, bone, adipose, viscera, neural, etc.).

### Example 5

Efficacy Experiments in Animals.

Animal efficacy experiments are conducted for candidate IS medications prior to human trials. In the instance of ultrapurified vancomycin, animal and human efficacy data with non-ultrapurified vancomycin preclude the need of doing further efficacy experiments in animals, though human efficacy experiments for this IS pharmaceutical are discussed below. For other candidate IS medications, a series of experiments are conducted in which standard models are used to test efficacy which mirror, as closely as possible, the human condition being treated by that drug. In the instance of an antibiotic, standard model infectious microorganisms are inoculated into standardized wounds prior to treatment with the IS antibiotic at the dose selected by the pharmacodynamics experiments previously performed. Model organisms are selected based on the known spectrum of coverage for that antibiotic. In the case of vancomycin-rifaximin, gram-positive and/or gram-negative microorganisms are inoculated into model wounds. These microorganisms potentially include but are not limited to: S. aureus strain Smith Diffuse, S. aureus strain SLC3, S. aureus methicillin-resistant strain Newman, S. aureas methicillin-resistant strain USA300, S. aureus vancomycin-resistant type vanA+, E. faecalis vancomycin-resistant type vanA+, S. pyogenes strain MGAS 158, P. aeruginosa strain LESB58, A. baumannii strain Ab5075, K. pneumoniae strain KPEN49, E. cloacae strain 21881, and E. coli K-12 MG1655. A preliminary calculation of IS medication concentration within the wound from the dose selected by the pharmacodynamics experiments is compared to known MIC values of model microorganisms (determined by broth microdilution assay), to be sure that bacterial eradication is expected from the administered dose. Following wound creation, microorganism inoculation, and subsequent IS antibiotic dosing, ongoing bacterial colonization with model organisms is tested using standard direct wound culture methods (plate colonization and turbidimetric assays), and/or real-time quantitative Polymerase Chain Reaction (PCR). IS doses of the candidate antibiotic are subject to dose escalation until microorganism colonization is undetectable. If this dose is higher than the close selected by the foregoing animal pharmacodynamics experiments, a further group of animals are included in the safety/pharmacodynamics experimental protocol at this new higher dose prior to human experiments.

### Example 6

Human Pharmacodynamics (Phase I) Trials.

All candidate IS pharmaceuticals will be subject to human pharmacodynamics trials. These trials involve approximately 10-100 patients divided into sequential dose-escalation groups, where dosing is based on wound surface area (with potential modification for wound tissues type composition). While dose selection is based on the results of foregoing animal experiments, the initial group of patients receive doses only a fraction of the safe and effective dose identified in animal experiments. After IS drug administration, wound and blood levels of the medication are measured at regular time points. The selection of these time points depends on the time course of action of the specific medication. In the instance of ultrapurified vancomycin, these time points will be directly following surgery, followed by repeat measurements at 24 hrs, 48 hrs, 72 hrs, and 96 hrs. These data points are used to establish the washout rate of ultrapurified vancomycin from the wound and determine if the drug is detectable in the blood stream. Patients are monitored for weeks after surgery for signs and symptoms of toxicity and/or side effects. When adverse events are not detected, a subsequent group of patients is enrolled at a higher dose per unit surface area of the wound. The process is repeated until adverse events are detected, signaling the safe dosing limit, or monitored drug levels within the wound significantly exceed the maximum conceivable therapeutic dose. In the case of ultrapurified vancomycin, such a level could be conceived of as 2-fold higher than the minimum to achieve wound drug levels in every tested patient high enough to reliably kill drug resistant microorganisms (>128 μg/mL of vancomycin). Such a high drug level would present a viable treatment for infections caused by drug resistant organisms and would also help to prevent the rise of drug resistant organisms from marginal drug concentrations. In the case of ultrapurified vancomycin and/or other ultrapurified IS medications, blood serum testing for endotoxins may or may not be required to demonstrate safety. An alternate strategy would be to monitor patients for signs and symptoms of endotoxemia after administration. In some instances, separate trials are conducted to study dosing for prevention as opposed to treatment of infections, which may have different effective dosing regimens. The results of these experimental trials guide dose-selection for subsequent safety and efficacy trials.

### Example 7

### Example 5

Human Safety and Preliminary Efficacy (Phase II) Trials.

All IS pharmaceutical candidates will require Phase II human safety and preliminary efficacy trials. These trials use approximately 300 patients in order to detect on the order of 1% adverse event rates. In the instance of ultrapurified vancomycin, ultrapurified vancomycin-rifaximin, or other IS antibiotics, the dose selected from the pharmacodynamics trial is administered to the wound in parallel with standard-of-care IV cephalosporin at recommended doses. This group is then compared to a similar sized group of patients who received standard-of-care perioperative IV cephalosporin, but no IS antibiotic. Patients are monitored until their wounds have completely healed for signs and symptoms of toxicity, side effects, or any other adverse events. Rates of wound infections after surgery are recorded and analyzed for differences from the control group though it must be noted that this trial design is not powered to detect significant changes in infection rates if the baseline rate is on the order of 1-2%. For this reason, the trial is conducted in patients at higher risk of infection but with very regularized wounds from surgery, spinal deformity patients, for example. When results of this trial indicate that the IS medication is safe for use at the selected dose, patients are enrolled in Phase III efficacy trials utilizing the same dose. If the dose is found to cause an unacceptable number of adverse events, a dose adjustment can be made and possibly the safety trial partially or completely repeated.

### Example 8

Human IS Medication Efficacy (Phase III) Trials.

All candidate IS medications will require human efficacy trials prior to approval for use via this new route of administration. In the instance of ultrapurified vancomycin and/or ultrapurified vancomycin-rifaximin efficacy trials use approximately 500-2500 patients to detect efficacy by reducing the rate of wound infections. However, given the theoretically highly improved safety profile of IS medications compared to their IV counterparts, along with the argument that IS antibiotics would create fewer drug resistant organisms than systemic administration of the same medication, a non-inferiority trial design would be adequate to merit regulatory approval. In such circumstances, an efficacy trial would require fewer patients to demonstrate non-inferior efficacy to standard of care IV antibiotics. In either case, patients are enrolled and randomized to receive IS ultrapurified antibiotic at the dose selected by the foregoing trials, IS ultrapurified antibiotic plus standard-of-care perioperative IV cephalosporin, or standard-of-care IV cephalosporin alone. Each patient is monitored for signs and symptoms of side effects, toxicities, other adverse events, wound complications, and especially wound infections, until wounds are completely healed. The standard CDC definitions of surgical site infections are used to document the presence of a wound infection after surgery. Statistical comparisons between the trial groups are made at regular intervals throughout the trial to determine if the trial should be stopped due to adverse events or if it should be stopped due to early clear detection of efficacy.

### EQUIVALENTS

Those skilled in the art will recognize, or be able to ascertain, using no more than routine experimentation, numerous equivalents to the specific embodiments described specifically in this disclosure. Such equivalents are intended to be encompassed in the scope of the following claims.

1-82. (canceled)

83. A method of reducing aerosolization of a lyophilized pharmaceutical composition having at least one low bioavailability therapeutic agent, the method comprising
wetting the lyophilized pharmaceutical composition having at least one low bioavailability therapeutic agent, wherein the wetting results in a paste but does not fully dissolve the pharmaceutical composition;
dissolving the paste into a solution;
emulsifying the solution with a metabolizable emulsifying agent; and
creating a gel comprising the emulsified solution, an aqueous solvent, and a metabolizable gelling agent;
wherein the gel is resistant to aerosolization.

84. The method of claim 83, wherein the emulsifying agent is lecithin.

85. The method of claim 83, wherein the gelling agent is non-proteinaceous.

86. The method of claim 83, wherein the gelling agent is a polysaccharide gelling agent.

87. The method of claim 86, wherein the polysaccharide gelling agent is selected from the group consisting of carbomers, poloxamers, and cellulose derivatives.

88. The method of claim 83, wherein the gelling agent comprises pluronic, lecithin, or isopropyl palmitate.

89.-95. (canceled)
